(19) United States
(12) Patent Application Publication
(10) Pub. No.: US 2026/0232848 A1
(43) Pub. Date: Aug. 13, 2026

(54) PET/MRI CONTRAST AGENTS FOR IN VIVO QUANTIFICATION OF EXTRACELLULAR ACIDIFICATION

(71) Applicant: Eberhard Karls Universität Tübingen Medizinische Fakultät Körperschaft des öffentlichen Rechts, Tübingen (DE)

(72) Inventors: André Ferreira Martins, Tübingen (DE); Jan Kretschmer, Tübingen (DE); Remy Chiaffarelli, Tübingen (DE); Yasmin Al-Moufleh, Reutlingen (DE)

(21) Appl. No.: 19/048,432

(22) Filed: Feb. 7, 2025

(51) Int. Cl. A61K 49/10; A61K 49/08

(52) U.S. Cl. CPC A61K 49/106; A61K 49/085; A61K 2123/00

(57) ABSTRACT

The disclosure provided a composition for imaging, comprising a compound of the general formula (I), wherein X is ¹⁸F, or ¹⁹F; Y is independently selected from —(CH₂)ₙ—COOH or —(CH₂)ₙ—COO(CH₂)ₘY¹, wherein n is 1, 2 or 3, m is 0, 1 or 2, and Y¹ is C1 to C5 linear or branched alkyl; and Z is —Z¹—NHSO₂—Z², Z¹ is linear or branched C1 to C5 alkyl or alkenyl, and Z² is substituted or unsubstituted alkyl, cyclyl or heterocyclyl; or a salt thereof; a Lanthanide compound; and optionally pharmaceutically acceptable excipients, wherein a Lanthanide complex is formed between the compound of formula (I) or salt thereof and the Lanthanide compound. In addition, a method of synthesis a composition for imaging, methods of using the composition for imaging, a method of using the composition evaluation of pH and/or perfusion a compound, and a method of synthesis a compound are provided.

### TECHNICAL FIELD

The present disclosure pertains to contrast agents for imaging. In particular, the present disclosure pertains to a composition for imaging, a method of synthesis a composition for imaging, methods of using the composition for imaging, a method of using the composition evaluation of pH and/or perfusion, a compound, and a method of synthesis a compound.

### BACKGROUND

It is known that many cancer cells exhibit a metabolic shift that favors increased glycolysis over mitochondrial oxidative phosphorylation, regardless of oxygen levels. This glycolytic upregulation leads to acidification of the extracellular pH (pHe) in the tumor microenvironment (TME). Extracellular acidification is characterized by a usually signifi-cantly lower extracellular pH (pH about 6.5 to 7.0) compared to normal tissues (pH about 7.4).

This phenomenon is due to the energy production of tumor cells, such as the Warburg effect, in which tumor cells rely preferentially on aerobic glycolysis. To maintain the slightly alkaline intracellular pH of normal tissues, these cells enhance several proton ex-trusion mechanisms, including the Na⁺/H⁺ exchanger, bicarbonate transporters, carbonic anhydrase IX, vacuolar ATPase, and H⁺/K⁺ ATPase. By actively exporting excess protons into the extracellular matrix, tumor cells cause the extracellular pH (pHe) of the tumor microenvironment (TME) to become acidic, measured as low as pHe 6.44 in certain tumors like the human MCF-7 breast carcinoma.

Tumor acidosis is closely associated with increased tumor growth rates, invasion, and metastasis. Despite the importance of pH and tumor acidosis, there is a lack of established tools for non-invasive, quantitative imaging of the spatial distribution of tumor pHe. Existing pH-responsive PET/MRI contrast agents are either kinetically unstable in vivo or difficult to synthesize with low radiochemical yields, limiting their practicality for robust preclinical and clinical use. It is therefore of general interest to quantitatively measure and track the pH in the tumor microenvironment.

US 2023/416457 A1 pertains to methods and compositions for imaging tumor acidosis using pH-activated near-infrared fluorescence probes. The described probes become fluorescent in acidic conditions, allowing for the visualization of tumor acidosis in vivo.

Frullano et al. (DOI: 10.1002/anie.201000075) shows an in vitro pH-responsive hybrid PET/MRI contrast agent. Synthesis is cumbersome and results in a very low radiochemical yield, making it impractical for widespread application. Pollard et al. (DOI: 10.3390/bios12020134) shows a pH sensor. The sensor exhibits kinetic instability, leading to the decomposition of the PET-radioactive co-agent during use, which compromises both imaging accuracy and patient safety.

Despite its critical role in tumor progression, no established techniques ex-ist for non-invasive, quantitative imaging of the spatial distribution of tumor pHe in the TME using a high-resolution imaging approach that can be used essentially in all tissues/organs.

It is therefore an object to provide compounds, precursors and compositions for multimodal imaging, as well as methods for their preparation, which allow imaging of the animal or human body with essentially no limit to the penetration depth, generally visualizing organs and tumors in internal or difficult to access body areas. Another object is to overcome the disadvantages associated with the prior art probes, such as their toxicity, concentration dependence, size, sensitivity to photobleaching or environmental conditions, including e.g. pH and oxidative compounds.

The present disclosure overcomes these limitations by introducing pH-responsive hybrid PET/MRI contrast agents that can be included into a composition for imaging as shown hereinafter. Agents have been designed that are easy to synthesize and allow for fast and efficient radiolabeling with a high radiochemical yield in the last synthesis step. These agents enable accurate measurement and imaging of extracellular pH within the tumor microenvironment, assisting physicians in tailoring treatment plans based on the specific pHe of a patient's tumor. This leads to improved therapeutic outcomes and minimizes un-necessary exposure to less effective drugs.

### SUMMARY

According to an aspect, provided is a composition for imaging, comprising a compound of the general formula (I):

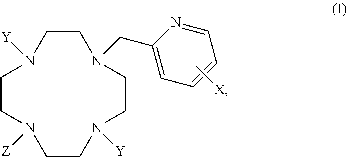

wherein
X is ¹⁸F, or ¹⁹F;
   Y is independently selected from —(CH₂)ₙ-COOH or —(CH₂)ₙ-COO(CH₂)ₘY¹, wherein n is 1, 2 or 3, m is 0, 1 or 2, and Y¹ is C1 to C5 linear or branched alkyl; and
   Z is —Z¹—NHSO₂—Z², Z¹ is linear or branched C1 to C5 alkyl or alkenyl, and Z² is substituted or unsubstituted alkyl, cyclyl or heterocyclyl;
      or a salt thereof;
      a Lanthanide compound; and
      optionally pharmaceutically acceptable excipients,
wherein a Lanthanide complex is formed between the compound of formula (I) or salt thereof and the Lanthanide compound.

The Lanthanide complex formed between the compound of formula (I) or salt thereof and the Lanthanide compound is hereinafter also referred to as the contrast agent or the agent. Specifically, the expression PET contrast agent or PET agent denotes the ¹⁸F radiolabeled Lanthanide complex (wherein X is ¹⁸F) and the expression MRI contrast agent or MRI agent denotes the ¹⁹F Lanthanide complex (wherein X is ¹⁹F).

The present disclosure provides pH-responsive hybrid PET/MRI contrast agents that are included into or constitute the present composition for imaging. These PET/MRI contrast agents have been found kinetically inert in vivo. The PET/MRI agents are easy to synthesize and allow for fast and efficient radiolabeling with a high radiochemical yield and enable accurate measurement and imaging of extracellular pH within the tumor microenvironment.

The disclosure thereby addresses a critical need in medical imaging by providing a kinetically stable, easily synthesized pH-responsive hybrid PET/MRI contrast agent with a high radiochemical yield. It overcomes significant limitations of existing technol-ogies, offers enhanced accuracy and reliability in tumor imaging and perfusion assessment. This advancement holds great potential for improving diagnostic capabilities and patient outcomes in oncology by enabling personalized treatment strategies based on accurate extracellular pH measurements.

By accurately identifying the extracellular pH of tumors, chemotherapy regimens can be tailored more effectively. For instance, acidic extracellular conditions can re-duce the efficacy of weakly basic drugs like daunorubicin, doxorubicin, and mitoxantrone by promoting their protonation, which decreases membrane permeability and cytotoxicity. Con-versely, weakly acidic chemotherapeutics such as chlorambucil, cyclophosphamide, and 5-fluorouracil exhibit increased cytotoxicity under acidic conditions. Therefore, the present contrast agent not only advances imaging technology but also plays a pivotal role in enhancing personalized medicine in oncology.

Specifically, the present agents provide
a robust stability, high kinetic inertness: unlike previous agents the present contrast agent remains stable in vivo, preventing decomposition and ensuring reliable imaging data. This renders in vivo use possible and enhances safety and reliability during imaging procedures;
simplified synthesis with high radiochemical yield: an efficient synthesis process with high radiochemical yield simplifies production and makes the agent more accessible for widespread use, particularly for clinical deployment;
improved imaging accuracy: by leveraging both PET and MRI modalities, the present agent minimizes inaccurate evaluations and provides an unprecedented accuracy in measuring and imaging extracellular pH and perfusion properties of tumors compared to standard contrast agents;
image-guided immunotherapies: the present agent is able to track extracellular pH in vivo, and it's the first of its kind able to monitor acidity in the tumor microenvironment and monitor immunotherapy response (Knopf et al., Molecular Cancer, 2024; https://doi.org/10.1186/s12943-023-01900-0); and
personalized treatment planning: accurate measurement of tumor pHe enables physicians to select the most appropriate chemotherapy regimens, improving patient outcomes and re-ducing exposure to ineffective treatments.

The present agents are generally highly feasible for imaging in vivo and determining the perfusion properties of tumors in vivo.

It is in principle possible to use a single-tracer approach wherein [¹⁸F]-q-pH1 is administered and PET/MRI imaging is performed simultaneously using the same system. The ¹⁸F isotope emits positrons, which are detected by the PET scanner, providing a direct, background-free molecular signal, that can be useful for quantifying tracer distribution or targeted delivery. The trivalent lanthanide ion, such as a Gd³⁺ ion, forming the metal center of the complex enhances MRI contrast in conventional proton-based MRI sequences.

Alternatively, and preferably, a dual-tracer approach is used wherein both compounds, [¹⁸F]-q-pH1 and [¹⁹F]-q-pH1, are administered. This strategy takes advantage of the distinct imaging properties of each isotope while maintaining a chemically similar structure. This setup is particularly useful for quantitative molecular imaging and multimodal functional assessments.

In the dual-tracer approach, [¹⁸F]-q-pH1 acts as the PET co-agent in a dual-modality PET/MRI contrast agent. The [¹⁸F] radiolabel provides PET imaging capabilities by emitting positrons, allowing for precise quantification. The [¹⁹F]-q-pH1 serves as the MRI co-agent, enabling responsiveness, which is observed as a change in the MRI signal. The lanthanide ion, such as the Gd³⁺ ion, forming the metal center of the complex enhances MRI contrast by T₁ weighted imaging, allowing for high-resolution anatomical localization. The dual-tracer approach provides matched pharmacokinetics and chemistry due to the al-most identical chemical structures, leading to improved quantification and validation despite the reduced concentration of the radioactive tracer or lower tracer doses.

T₁ weighted imaging is an MRI technique that primarily reflects differences in T₁ relaxation times of tissues. T₁ relaxation time is the time it takes for protons to realign with the external magnetic field after being disturbed by a radiofrequency (RF) pulse. R₁ relaxivity is the inverse of T₁ relaxation time. R₁ relaxivity of the MRI co-agent is pH dependent, because the protonation of the sulfonamide group can lead to the dissociation of ligand arm bearing the sulfonamide group from the lanthanide core, increasing the R₁ relaxation rate of the surrounding water. A known ratio of MRI co-agent to PET co-agent allows trans-lating the radioactivity detected by the PET detector into the concentration of the MRI co-agent. This known concentration and the R1 relaxation rate permits calculating the r1 relaxivity and determining pH using a calibration curve of pH versus r1 relaxivity for the MRI co-agent.

Lanthanide complexes, such as those based on Dy and Ho, have shown to be effective T₂ MRI contrast agents due to their strong paramagnetic properties. These complexes may induce local magnetic field inhomogeneities, leading to enhanced transverse (T₂) relaxation effects, which improve the contrast in T₂ weighted imaging. The lanthanide ions, such as Dy³⁺ and Ho³⁺, may efficiently decrease T₂ and T₂* relaxation times, making them particularly useful in detecting lesions with increased water content, such as tumors, edema, or hemorrhagic regions. Furthermore, T₂* imaging is particularly sensitive to local susceptibility effects, allowing for enhanced detection of microhemorrhages or calcifications, which may not be as visible in conventional T₂ weighted images.

Both T₂ and T₂* imaging techniques assess transverse relaxation properties of tissues, but they differ in their sensitivity to magnetic field inhomogeneities. T₂ imaging measures the decay of transverse magnetization due to interactions at the molecular level, known as spin-spin relaxation. In contrast, T₂* imaging captures the combined effects of spin-spin relaxation and magnetic field inhomogeneities, resulting in a faster decay of the transverse magnetization. This is characterized by the T₂* relaxation time, which is generally shorter than the T₂ time.

In general, both T₁ weighted and T₂-weighted MRI sequences can be performed to obtain complementary diagnostic information. While T₁ weighted imaging provides anatomical details and is preferably used with a Lanthanide complex, T₂ and T₂* imaging enhance visualization of pathological tissue by highlighting differences in water content and magnetic susceptibility. Certain lanthanide complexes designed for T₂ contrast can represent an alternative to conventional lanthanide based agents, particularly for applications where T₂ contrast is more diagnostically relevant. For instance, Dy and Ho complexes can be expected to generate a strong T₂ weighted contrast while maintaining biocompatibility.

A plurality of different in vivo Magnetic Resonance Imaging (MRI) techniques can be used for visualizing and/or assessing biological tissues and functions. Examplary and preferred techniques include Diffusion-Weighted Imaging (DWI), which measures the diffusion of water molecules within tissues to detect abnormalities such as acute ischemic stroke, functional MRI (fMRI), which monitors brain activity by detecting changes in blood oxygenation and flow, Magnetic Resonance Spectroscopy (MRS), which analyzes the chemical composition of tissues to identify metabolic changes associated with diseases, Perfusion Imaging, which evaluates blood flow within tissues to assess conditions like tumors or ischemic areas, and Real-Time MRI, which captures dynamic processes such as cardiac function in real-time.

Expressions such as lanthanide compound or lanthanide complex are di-rected to a metal ion from a metal of the lanthanide group forming a compound or complex. Accordingly, the expressions lanthanide compound or lanthanide complex are not limited to Lanthanum (La) but can include any of Lanthanum (La), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium I, Ytterbium (Yb), and Lutetium (Lu). Hence, a lanthanide compound or lanthanide complex can be a metal compound or metal complex, wherein the metal is selected from the lanthanide group, specifically any of Lanthanum (La), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium I, Ytterbium (Yb), and Lutetium (Lu).

Examples of suitable lanthanide compounds include Gd (Gadolinium), Dy (Dysprosium), Europium (Eu), Ho (Holmium), Tb (Terbium), and Tm (Thulium), preferably Gd (Gadolinium), Dy (Dysprosium), and Ho (Holmium), more preferably Gd (Gadolinium). The Lanthanide compound is preferably used as a trivalent salt, particularly a trivalent salt of Gd³⁺, Dy³⁺, Eu³⁺, Ho³⁺, Tb³⁺, and Tm³⁺, with the counterions as described for Gd³⁺ hereinafter, such as GdCl₃.

According to an aspect, X is in ortho or para position to N. The para position is preferred.

According to an aspect, Y is —CH₂—COOH or —CH₂—COOCH₃.

According to an aspect, Z¹ is —CH₄— or —CH₂—CH₄—.

According to an aspect, Z² is phenyl having a single functional group in para position.

According to an aspect, Z² is 4-methoxy-phenyl.

According to another aspect the Lanthanide complex formed between the compound of formula (I) is

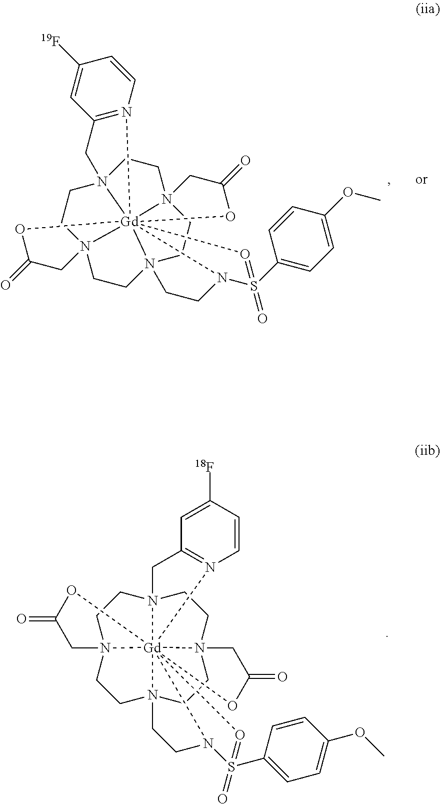

The compound (iia) is also denoted MRI co-agent [¹⁹F]-q-pH1, [¹⁹F]-q-pH1, or [Gd([¹⁹F]q-pH-1)], eventually also without indicating the isotope. The compound (iib) is also denoted PET co-agent [¹⁸F]-q-pH1, [¹⁸F]-q-pH1, or [Gd([¹⁸F]q-pH-1)]. Compounds (iia) and (iib) together can be denoted q-pH1 or [¹⁸F]/[¹⁹F]-q-pH1.

According to an aspect, the Lanthanide compound is GdCl₃.

According to another aspect, the composition for imaging comprises the compound of the general formula (II), wherein X is ¹⁹F, and the compound of the general formula (II), wherein X is ¹⁸F, at the same time. These two compounds of the general formula (II) can be present in the composition in different molar ratios as indicated hereinafter. This has the advantage of reduced tracer doses.

According to another aspect, provided is a method of synthesis a composition for imaging. The method comprises the steps of:

providing a compound of the general formula (I)

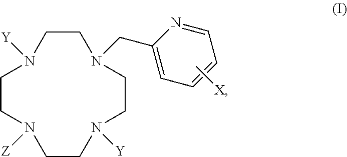

wherein
X is N⁺(CH₃)₃, NO₂ or ¹⁹F;
   Y is independently selected from —(CH₂)ₙ—COOH or —(CH₂)ₙ-COO(CH₂)ₘY¹, wherein n is 1, 2 or 3, m is 0, 1 or 2, and Y¹ is C1 to C5 linear or branched alkyl; and
   Z is —Z¹—NHSO₂—Z², Z¹ is linear or branched C1 to C5 alkyl or alkenyl, and Z² is substituted or unsubstituted alkyl, cyclyl or heterocyclyl;
   or a salt thereof, optionally wherein, if X is N⁺(CH₃)₃ or NO₂, the compound of the general formula (I) or the salt thereof is reacted with a ¹⁹F-salt; and
   b) reacting the compound of the general formula (I) or salt thereof a with a Lanthanide compound, thereby forming a Lanthanide complex of the compound of general formula (I) or salt thereof;
   optionally, c) radiolabeling the Lanthanide complex from step b) with ¹⁸F.

Upon reacting the compound of the general formula (I) with a trivalent Lanthanide ion, such as Gd³⁺, e.g. GdCl₃, a stable, low-molecular weight MRI hybrid contrast agent, such as [¹⁹F]q-pH-1, is obtained, provided that R is ¹⁹F. [¹⁹F]q-pH-1 can be prepared directly from the [¹⁹F] precursor or from the the compound of the general formula (I), wherein R is NO₂.

The compound of the general formula (I), wherein R is a N⁺(CH₃)₃ or nitro moiety, is easy to handle and represents a precursor of the compounds of general formula (I), wherein R is ¹⁸F or ¹⁹F, and the corresponding Lanthanide complexes.

The compound of the general formula (I), wherein R is N⁺(CH₃)₃, represents a salt of the compound of the general formula (I). Suitable counterions/anions are known to the art and preferably include pharmaceutically acceptable ions. Examplary anions to R=N⁺(CH₃)₃ and/or anions used in a pharmaceutically acceptable salt include one or more of chloride, nitrate, acetate, triflate, sulfate, phosphate, and hydrogen phosphate.

Subjecting the ¹⁹F labeled compound, such as [¹⁹F]q-pH-1, further to fluorine isotopic exchange, optionally and preferably in the last synthesis step, yields the corresponding ¹⁸F labeled compound, such as [¹⁸F]q-pH-1, in amounts sufficient allowing the application of PET. Alternatively, the compound of the general formula (I), wherein R is N⁺(CH₃)₃ or nitro, can be subjected to nucleophilic aromatic substitution of the nitro group with ¹⁸F. Tested in healthy animals, the ¹⁸F and ¹⁹F labeled compounds of the general formula (I), such as [¹⁸F]q-pH-1 and [¹⁹F]q-pH-1, enables spatial mapping of pHe and perfusion assessment in organs/tissues, such a mouse kidneys, via PET and MRI, providing a powerful, comprehensive tool to characterize acidosis in tissues. In principle, the ¹⁸F or ¹⁹F labeled compounds of the general formula (I) alone, such as [¹⁸F]q-pH-1 or [¹⁹F]q-pH-1, can be used for perfusion assessment

Both compounds, the ¹⁹F labeled Lanthanide complex, such as [¹⁹F]q-pH-1 used for MRI and the ¹⁸F labeled Lanthanide complex, such as [¹⁸F]q-pH-1, used for PET are structurally highly similar. Thereby the ¹⁸F and ¹⁹F labeled Lanthanide complexes complement each other and permit multimodal imaging.

The present Lanthanide complexes, such as [¹⁸F]q-pH-1 and [¹⁹F]q-pH-1, exhibit a very low toxicity in spite of the Gadolinium rendering them suitable for use in vivo, without negatively affecting the subject/patient, who was administered the present composition for imaging, optionally in a therapeutically effective amount.

The skilled person is generally aware about producing the claimed compounds and compositions.

For instance, the synthesis of the chelate compound, a derivative of DOTA (2,2′,2″,2′″-(1,4,7,10-Tetraazacyclododecane-1,4,7,10-tetrayl)tetraacetic acid) and the corresponding Lanthanide complexes therefrom can involve preparing the DOTA ligand by alkylating cyclen with bromoacetic acid to yield the tetraacetic acid derivative; and reacting the ligand with lanthanide ions, such as Gd³⁺ ions, under controlled conditions, for instance in polar solvents, including water or methanol. General information about synthesis and syn-thetic strategies can be derived from Borbas, K. E. et al., Trivalent Gd-DOTA Reagents for Modification of Proteins, RSC Adv. 2015, 5, 51629-51640. DOI: 10.1039/C5RA20359G; and Yu, M. et al., Design and Synthesis of Novel DOTA (Gd³⁺), Polymer Conjugates as Potential MRI Contrast Agents, J. Mater. Chem. 2011, 21, 2565-2573. DOI: 10.1039/C1JM00005E, the contents of each of which are incorporated herein by way of reference in their entirety.

Recent approaches involve solid-phase synthesis (SPS) as efficient methods for creating lanthanide complexes, such as gadolinium (Gd) complexes, with DOTA derivatives. This approach simplifies the process of constructing complex structures by immobi-lizing intermediates on a solid support, enabling streamlined purification and functionaliza-tion. An example involves the synthesis of DOTA-conjugated bombesin derivatives directly on a resin, where the DOTA moiety is coupled to a tumor-targeting peptide in a stepwise fashion. This method allows for efficient radiolabeling with isotopes (Blasi et al., EJNMMI Res. 2019, 9, 39. DOI: 10.1186/s13550-019-0539-0, the contents of each of which is incorporated herein by way of reference in its entirety).

Reference is also made to the following documents showing for details about the synthesis of the compounds of the general formula (I) with and without complexed Gadolinium, the contents of which are included by way of reference in their entirety. EP 22 212 471 A1; “A Macrocyclic Hybrid PET/MRI Probe for Quantitative Perfusion Imaging In Vivo”. J. Kretschmer, R. Chiaffarelli, J. Cotton, J. Blahut, J. Ráliš, M. Dračínský, S. Matějková, M. Vuozzo, U. Seeling, A. M. Schmid, A. F. Martins and M. Polasek, Angew. Chem., Int. Ed., 2024, 10.1002/anie.2024095; Pollard et al. (DOI: 10.3390/bios12020134)

Step b) can be achieved by reacting the compound of general formula (I) with a Lanthanide compound, specifically a Gd³⁺ ion, thereby forming a Gd(II) complex of the compound of general formula (I).

The conditions of the complexation reaction may be as follows. A solution containing trivalent lanthanide ions, such Gd³⁺ ions, in the form of salt, e.g., chloride, bromide, sulfate, or nitrate, is mixed with a solution of the compound of general formula (I), such as in molar ratio of the trivalent lanthanide ion, such as the Gd³⁺ ion, to compound of general formula (I) in the range of from 1:0.5 to 1:100, such as from 1:0.7 to 1:50, or 1:0.9 to 1:10. Concentrations of the soluble components may be selected from the concentration range permitted by solubility of such compounds in a given solvent at a given temperature, such as in the concentration range 10⁻⁶-0.5 mol/L. The solvent may be water, a water-miscible organic solvent such as methanol, ethanol, propanol, isopropanol, acetone, acetonitrile, or a mixture thereof. An organic or inorganic base, such as LiOH, NaOH, KOH, aqueous NH3, triethylamine, N,N-diisopropylethylamine or pyridine, is added to the reaction mixture in order to compensate for protons released during the complexation, and the complexation takes place in the solution. From 1 to 10 molar equivalents of base may be added per molecule of the compound of the general formula (I). Eventually, the reaction can take place in a buffer. In such case there is no need of adding organic or inorganic base to the reaction mixture. The mixture can be stirred or shaken at room temperature or elevated temperature for up to 24 hours to afford complete complexation. For instance, the mixture is stirred or shaken at 40° C. for 15 minutes. A reasonable excess of the compound of general formula (I) may be used to accelerate the complexation and to shift the equilibrium towards formation of the chelates. The resulting lanthanide(III) complex, such as a Gd(III) complex, of the compound of general formula (I) may optionally be desalted and purified from excess of the compound of general formula (I) and other impurities.

Purification may be achieved, for example, by column chromatography or high-performance liquid chromatography (HPLC). Preferably, the chromatographic separation is achieved using HPLC on C8, C18 or phenyl-hexyl reversed phase. The mobile phase used for chromatographic purification may contain water and 3-40 vol. % of methanol, ethanol or acetonitrile. Optionally, 0.01-0.1 mol/L of a buffer is used in the mobile phase, wherein the buffer comprises ammonium format pH=7.0 or ammonium acetate pH=7.0. Fractions containing the desired metal chelate are collected and evaporated or lyophilized.

The optional step c) of radiolabeling the Lanthanide complex from step b) can be performed with ¹⁸F by using either ¹⁹F to ¹⁸F isotopic exchange on the pyridine moiety of the Lanthanide complex, wherein R is F; or by using nucleophilic aromatic substitution of the nitro group with ¹⁸F on the pyridine moiety of the Lanthanide complex, wherein R is NO₂.

The following conditions can be used for performing step c). The lanthanide chelate, such as Gd(III) chelate, from step b) is dissolved in a solvent, such as acetone, acetonitrile, dichloromethane, N,N-dimethylformamide (DMF), N,N-dimethylacetamide (DMAC), dimethylpropyleneurea (DMPU), dimethylsulfoxide (DMSO), sulfolane, ethyl acetate, hexamethylphosphoramide (HMPA), pyridine, tetrahydrofuran (THF), methanol, ethanol, propanol, isopropanol, butanol, tert-butanol, N-methyl-2-pyrrolidone (NMP), 1,3-dime-thyl-2-imidazolidinone, tert-amyl alcohol, water or a mixture thereof. The final concentration of Lanthanide complex in the solvent or solvent mixture can be in the range of from 10⁻¹² M to 2 M. Optionally, a source of non-radioactive ¹⁹F-ions is added to achieve a concentration in the range of from 10⁻¹² M to 2 M. The source of non-radioactive ¹⁹F-ions may be a ¹⁹F salt, such as tetramethylammonium fluoride, tetraethylammonium fluoride, tetrapropylammonium fluoride, tetrabutylammonium fluoride, tetrahexylammonium fluoride, NaF, KF, RbF, CsF, [Na(15-crown-5)]F, [K(18-crown-6)]F, [Cs(21-crown-7)]F, [Na(cryptand[2.2.1])]F, [K(cryptand[2.2.2])]F or [Cs(cryptand[322])]F. The source of non-radioactive F-ions is added in order to achieve a nucleophilic aromatic substitution of NO₂ group with ¹⁹F. If the lanthanide chelate, such as the Gd(III) chelate, from step b) does not contain any NO₂ group, then addition of non-radioactive F-ions can be omitted.

The source of radioactive ¹⁸F ions can be added in an amount that corresponds to the desired activity. In terms of molar equivalents, this represents only a fraction of the amount of the lanthanide chelate, such as the Gd(II) chelate, and/or of the non-radioactive F ions. Suitable amounts (activity) of ¹⁸F-ions are known to the skilled person. The source of radioactive ¹⁸F ions can correspond to the sources of non-radioactive F ions listed above (e.g. [¹⁸F]tetramethylammonium fluoride, [¹⁸F]tetraethylammonium fluoride, [¹⁸F]tetrapropylammonium fluoride, [¹⁸F]tetrabutylammonium fluoride, [¹⁸F]tetrahexylammonium fluoride, Na¹⁸F, K¹⁸F, Rb¹⁸F, Cs¹⁸F, [Na(15-crown-5)]¹⁸F, [K(¹⁸-crown-6)+]¹⁸F, [Cs(21-crown-7)]¹⁸F, [Na(cryptand[2.2.1])]¹⁸F, [K(cryptand[22.2])]¹⁸F, or [Cs(cryptand[3.22])]¹⁸F). The reaction mixture is then stirred at a temperature in the range of from −50° C. to 250° C., preferably in the range of from 5° C. to 200° C., for at least 0.1 s, preferably for at most 8 hours.

Step c) may be performed in the solvent selected from acetonitrile, DMF, DMAC, DMPU, DMSO, sulfolane, HMPA, THF, tert-butanol, NMP, 1,3-Dimethyl-2-imidazolidinone, tert-amyl alcohol, water or a mixture thereof. The final concentration of lanthanide chelate, such as Gd(III) chelate, in the solvent or solvent mixture is in the range of from 10⁻⁶ M to 0.5 M. Optionally, a source of non-radioactive F-ions is added to achieve a concentration in the range of from 10⁻⁶ M to 0.5 M. The source of non-radioactive F ions can be selected from tetramethylammonium fluoride, tetrabutylammonium fluoride, NaF, KF, CsF, [Na(15-crown-5)]F, [K(18-crown-6)]F, [Na(cryptand[2.2.1])]F, and [K(cryptand[2.2.2])+]F. A source of radioactive 18F ions is added, in the amount that corresponds to the desired activity. The source of radioactive 18F ions corresponds to the sources of non-radioactive F ions listed above. The reaction mixture can be stirred at a temperature in the range of from 25° C. to 200° C. for at least 1 minute, preferably for at most 2 hours.

Optionally, the solvent is selected from acetonitrile, DMF, DMSO or mixtures thereof. The final concentration of Gd(III) chelate is in the range of from 0.1 mM to 100 mM. Optionally, a source of non-radioactive F− ions is added to achieve a concentration in the range of from 1 mM to 300 mM. The source of non-radioactive F− ions is preferably selected from tetrabutylammonium fluoride or [K(cryptand[2.2.2])+]F−. A source of radioactive 18 F-ions is added, in the amount that corresponds to the desired activity. The source of radioactive 18 F-is selected from [18 F]tetrabutylammonium fluoride and [K(cryptand[22.2])+]18 F-. The reaction mixture is then stirred at a temperature in the range of from 25° C. to 150° C. for at least 1 minute, preferably for at most 100 minutes.

The skilled person is well aware about selecting suitable pharmaceutically acceptable excipients. Suitable pharmaceutically acceptable excipients can be selected from the group containing solvents, such as an aqueous solution or a saline solution, buffers, such as phosphate buffer or HEPES, ionization additives, antioxidants, and antimicrobial additives. Further pharmaceutically acceptable excipients may include vinyl polymers, polyoxy-ethylene-polyoxypropylene polymers or co-polymers thereof, polysaccharides, proteins, polyethyleneoxide, and acrylamide polymers and derivatives or salts thereof. It is understood that polyethyleneoxide includes polyethylene glycol. The vinyl polymers may be selected from the group consisting of polyacrylic acid, polymethacrylic acid, polyvinyl pyrrolidone and polyvinyl alcohol. The polysaccharides may be selected from the group consisting of cellulose or cellulose derivatives, glycosaminoglycans, agar, pectin, alginic acid, dextran, starch and chitosan. The glycosaminoglycans may be selected from the group consisting of hyalu-ronic acid, chondroitin, and related molecules. The proteins may be selected form the group consisting of collagen, gelatin and fibronectin.

Further details on the synthesis on the composition of imaging and its precursor, the compound of the general formula (I). as well as the synthesis thereof will be provided hereinafter with respect to the compounds of the general formula (IIa) and (Ilb), which may be generally employed.

According to another aspect, provided is a method of using the present composition for imaging, comprising the steps of:
(a) administering the composition to a subject in need thereof;
(b) detecting the Lanthanide complex; and
(c) forming an image therefrom.

The present composition for imagining comprises a coordination compound of a trivalent lanthanide ion, such as a Gd(III) ion, and the cyclen based compound of general formula (III) as defined hereinafter. The presence of said coordination compound, containing lanthanide(II), such as Gd(III), and 19 F, such as the compound of the general formula (IIa), provides for the MRI signal, while the presence of the compound containing lanthanide (II), such as Gd(III), and ¹⁸F provides for the PET signal. As the amount of ¹⁸F radio-nuclide needed for the PET signal is generally lower than the amount of lanthanide chelate, such as the Gd(III) chelate, needed for the MRI contrast, MRI contrast of the pharmaceutical formulation may be enhanced by the presence of non-radioactive lanthanide chelate, such as Gd(III) chelate, of the compound of general formula (II), wherein X is 19F, such as (Ila).

It is preferred that the radioactive and non-radioactive lanthanide chelate, such as Gd(III) chelate, differs only in the fluorine isotope (meaning that the coordination compounds are structurally identical, differing only in whether bearing ¹⁹F or ¹⁸F isotope) for providing the same pharmacokinetics. The concentrations of the non-radioactive lanthanide chelate, such as Gd(III) chelate, should be sufficient to achieve MRI contrast, the concentration of the PET contrast agent should be sufficient to achieve PET contrast. These concentrations are known to the skilled person. The molar ratio of non-radioactive lanthanide chelate, such as Gd(III) chelate, such as the compound of the general formula (IIa), to the PET contrast agent, such as the compound of the general formula (IIb) can be from about 0.1 to about 10.0.

According to an aspect, the composition is used for imaging an extracellular pH.

According to an aspect, the composition is used for imaging perfusion.

According to an aspect, the imaging is magnetic resonance imaging (MRI) and/or positron emission tomography (PET). Optionally, the imaging can include single photon emission computed tomography (SPECT).

According to an aspect, magnetic resonance imaging (MRI) includes at least one of T₁ weighted imaging, T₂ weighted imaging, and T₂* weighted imaging.

According to an aspect, the subject is a vertebrate, a mammal or human.

According to an aspect, the composition is administered orally, systemically, via cerebral spinal fluid injection, vein injection, muscle injection, or peritoneal injection.

The dosage form of the pharmaceutical preparation is usually a form for administration by injection, most often as a bolus or as an infusion, preferably intravenously.

According to an aspect, the imaging is in vivo.

According to an aspect, the composition is used for detecting and/or monitoring tumor progression in vivo.

According to an aspect, the composition is used for detecting and/or monitoring one or more of ischemia, stroke, and an inflammatory disease in vivo. Examples of inflammatory diseases include rheumatoid arthritis, cancer-associated inflammation, sepsis and system inflammatory response syndrome, chronic obstructive pulmonary disease, asthma, inflammatory bowel disease, osteoarthritis, gout, chronic kidney disease, uremic acidosis, periodontitis, and multiple sclerosis. Said conditions are also characterized by pH shifts in the cellular environment rendering them promising targets for the present compositions and methods.

According to an aspect, provided is a method of using the composition of claim 1 for magnetic resonance imaging (MRI) and/or positron emission tomography (PET) evaluation of pH and/or perfusion, comprising the steps of:
(a) contacting a target with the composition;
(b) detecting the Lanthanide complex; and
(c) measuring the amount and/or concentration of the Lanthanide complex in the target
(d) evaluating the pH and/or the perfusion.

According to another aspect, provided is a method of using the present composition of claim for imaging, comprising the steps of:
(a) contacting a target with the composition;
(b) detecting the Lanthanide complex; and
(c) measuring the amount and/or concentration of the Lanthanide complex in the target.

According to an aspect, provided is a compound of the general formula (I)

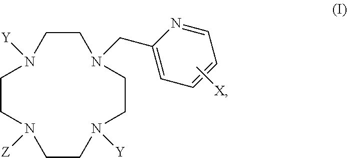

X is N⁺(CH₃)₃, NO₂, ⁸F, or ¹⁹F; Y is independently selected from —(CH₂)ₙ—COOH or —(CH₂)ₙ—COO(CH₂)ₘY¹, wherein n is 1, 2 or 3, m is 0, 1 or 2, and Y¹ is C1 to C5 linear or branched alkyl; and Z is —Z¹—NHSO₂—Z², Z¹ is linear or branched C1 to C5 alkyl, and Z² is substituted or unsubstituted alkyl, cyclyl or heterocyclyl; or a salt thereof.

Optionally, X is in ortho or para position to N. The para position is preferred. The compound exhibits high stability to in vivo degradation, resulting in low toxicity for in vivo imaging.

Optionally, Y is —CH₂—COOH or —CH₂—COOCH₃. The compounds exhibits high stability to in vivo degradation, resulting in low toxicity for in vivo imaging. Still optionally, Y is —CH₂—COOH.

Optionally, Z¹ is —CH₄— or —CH₂—CH₄—. This provides the sulfonamide residue with sufficient distance from the cyclen moiety, sufficient mobility and steric arrangement for coordination to the lanthanide ion, such as Gd ion, of the Lanthanide complex. Still optionally, Z¹ is —CH₄—.

Z² is not particular limited but can be a substituted or unsubstituted alkyl, cyclyl or heterocyclyl residue, wherein the cyclyl and heterocycly residue, respectively, has a single ring structure. Examples include cyclopentyl, cyclopentadienyl, cyclohexanyl, benzyl, pyridinyl, pyranyl, or thiopyranyl. Substituents can be located in ortho or para position with respect to the sulfonamide residue and include a linear or branched alkoxyl residue with 1 to 5 C-atoms, a nitro residue, a carboxyl residue, or an alkylester residue with 1 to 5 C-atoms. The substituted or unsubstituted alkyl alkyl residue can be a linear or branched alkyl residue with 2 to 5 C-atoms. A substituted alkyl residue can include a single substituent, such as a linear or branched alkoxyl residue with 1 to 5 C-atoms, a nitro residue, a carboxyl residue, or an alkylester residue with 1 to 5 C-atoms.

Optionally, the substituted or unsubstituted cyclyl or heterocyclyl moiety does not have reactive moieties, such as amino or hydroxy moieties, that can lead to decomposition.

Optionally, Z is 4-methoxy-phenyl, 4-ethoxy-phenyl, 4-nitro-phenyl, 4-amino-phenyl, 4-hydroxy-phenyl, 4-carboxy-phenyl, or 4-phenyl-formate.

Gadolinium (Gd) compounds that can be used for the composition for imaging are not particularly limited and include trivalent Gd-salts, such as GdCI₃.

According to another aspect, provided is a method of synthesis a compound of the general formula (I):

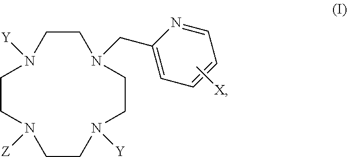

wherein
X is N⁺(CH₃)₃, NO₂, ¹⁸F, or ¹⁹F;
Y is independently selected from —(CH₂)ₙ-COOH or —(CH₂)ₙ-COO(CH₂)ₘY¹, wherein n is 1, 2 or 3, m is 0, 1 or 2, and Y¹ is C1 to C5 linear or branched alkyl; and
Z is —Z¹—NHSO₂—Z², Z¹ is linear or branched C1 to C5 alkyl, and Z² is substituted or unsubstituted alkyl, cyclyl or heterocyclyl;
or a salt thereof;
   the method comprising the steps of:
reacting a compound of the general formula (III)

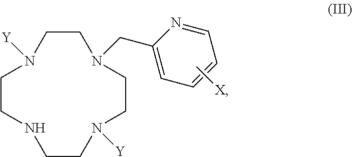

or a salt thereof
with A-Z¹—(NH)SO₂—Z², wherein A is configured to perform a nucleophilic substitution reaction with a secondary amine;
optionally wherein A-Z¹—(NH)SO₂—Z² is prepared by nucleophilic substitution of A-Z¹—(NH)-A¹ and A²-SO₂—Z², wherein A¹ and A² allow a mutual nucleophilic substitution reaction.

According to an aspect, X is in ortho or para position to N. The para position is preferred.

According to an aspect, Y is —CH₂—COOH or —CH₂—COOCH₃.

According to an aspect, Z¹ is —CH₄— or —CH₂—CH₄—.

According to an aspect, Z² is phenyl having a single functional group in para position.

According to an aspect, Z² is 4-methoxy-phenyl.

These and other objects and advantageous aspects will become apparent from a description of exemplary embodiments, with reference to the following figures.

### BRIEF DESCRIPTION OF THE DRAWINGS

FIG. 1 shows the hybrid PET/MRI contrast agent q-pH1.

FIG. 2 schematically shows pH dependency of the MRI-coagent [¹⁸F]-q-pH1.

FIG. 3 schematically shows the formation of the radiolabeled PET tracer [¹⁹F]-q-pH1.

FIG. 4 shows a chromatogram of the [Gd(NO₂-q-pH-1] radiolabeling experiment.

FIG. 5 shows longitudinal relaxation rates and MRI imaging of [Gd([¹⁹F]q-pH-1)].

FIG. 6 A-D show the pH-dependent change in longitudinal relaxation rates and MR imaging of [Gd([¹⁹F]q-pH-1)].

FIG. 7 shows the transversal relaxation rates and MR imaging of [Gd([¹⁹F]q-pH-1)].

FIG. 8 A-D show the pH-dependent change in transversal relaxation rates and MR imaging of [Gd([¹⁹F]q-pH-1)].

FIG. 9 shows in-vitro studies of q-pH-1.

FIG. 10 shows q-pH-1 evaluation of different kidney regions in laboratory mice.

FIG. 11 shows kidneys perfusion evaluation using the present hybrid PET/MRI pH contrast agent.

FIG. 12 shows in vivo pH evaluation.

FIG. 13 shows the in-vivo PET pharmacokinetics of q-pH-1 in healthy mouse.

### EMBODIMENTS

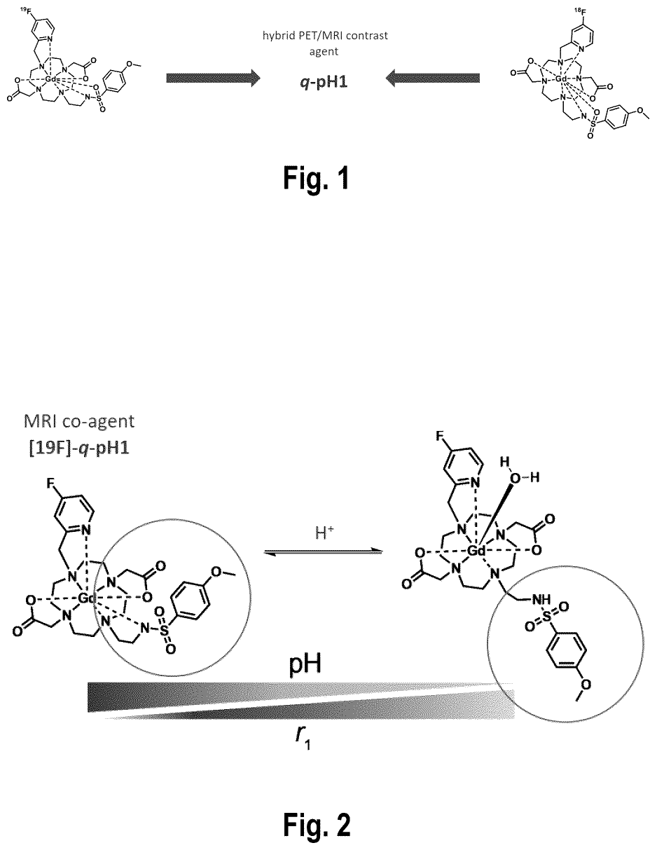

In the following the present compositions for imaging, specifically their synthesis and in-vivo characteristics, are exemplified with respect to the compounds (Ila) and (Ilb) forming the hybrid PET/MRI contrast agent q-pH1 as shown in FIG. 1.

The hybrid PET/MRI contrast agent q-pH1 constitutes the first in vivo stable PET/MRI pH responsive probe with efficient radiolabeling, in which the MRI co-agent [¹⁸F]-q-pH1 is for quantification purposes. Both agents are chemical identical with exception to the differing fluorine isotope providing them the same pharmacokinetic properties.

As can be seen from the schematic view of FIG. 2, the coagent [¹⁸F]-q-pH1, specifically the sulfonamide ligand arm, is pH dependent. At lower pH, sulfonamide pendat armis released from the Lanthanide complex in favor of a water molecule that is co-ordinated to the Gd ion. The sulfonamide pendant arm decoordinates from the metal center at a low pH, allowing access to a water molecule, leading to higher relaxivity. This process is reversible and a higher pH favors coordination of the sulfonamide ligand arms.

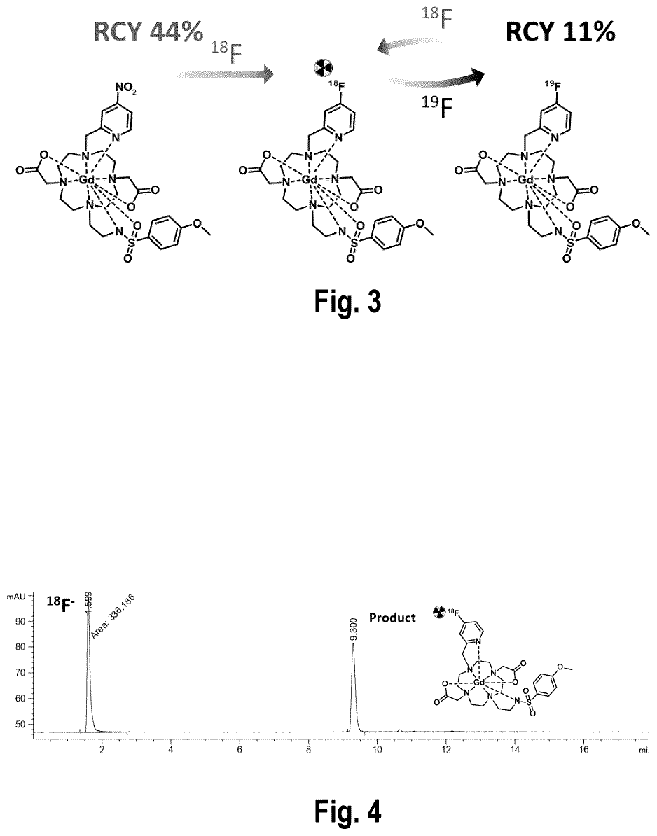

With further reference to FIG. 3, the formation of the radiolabeled PET tracer [¹⁸F]-q-pH1 from the corresponding nitro and non-radioactive fluorine precursors is shown. [¹⁸F]-q-pH1 can be prepared in less than 30 minutes by nucleophilic substitution from the nitro precursors and by isotopic fluorine exchange on a fluoro precursor with the indicated radiochemical yield (RCY). Both options offer a sufficient RCY, allowing them to be used in simple facilities, such as hospitals, and in the final synthesis step, just prior to use. Hybrid PET/MRI probe for in vivo quantification of pH

RADIOLABELING EXPERIMENTS: A solution containing cyclotron produced [¹⁸F]fluoride was absorbed on the QMA column (Sep-Pak, Waters) in OTf-cycle. Pre-conditioning of the column to the OTf-cycle was done by washing the QMA column with KOTf (0.48 M, 10 mL) followed by washing with H₂O (10 mL), air (10 mL) and blowing dry with 20 mL of air. Elution of [¹⁸F]F- from OTf-preconditioned column was done by solution of TBAOTf (5 mg) in dry methanol. The precursor for radiolabeling was introduced in DMSO.

MRI IMAGING: Phantom MR imaging was acquired on a 7 T preclinical MR scanner (Bruker Clinscan, Bruker BioSpin, Ettlingen, Germany) using an 86-mm diameter ¹H transceiver volume coil (Bruker). temperature-controlled holders for PET and MR scanners. The 3D-printedABS plastic phantom with inlets fitted for 0.3 mL tubes is prepared with heated 2% agarose gel and left to solidify before sampling.

### Pet/Mri Imaging:

MRI SEQUENCES: Two series of samples were prepared for each contrast agent. The first phantom series of varying concentration (0.1, 0.2, 0.3, 0.4 and 0.5 mM) and of constant pH 7.4 were dissolved in different environments such as 10 mM HEPES buffer, 0.6 mM human serum albumin or human serum (Sigma-Aldrich) and prepared in 0.3 mL Eppendorf tubes. In a second series, 6 samples with a constant concentration of 0.5 mM were adjusted to varying pH-levels (pH 5.0, 6.0, 6.5, 7.0, 7.4, 8.0) using sodium hydroxide and hydrochloric acid. The second series was also dissolved in the environments mentioned previously and sampled into 0.3 mL Eppendorf tubes.

THE following sequences were acquired:
T₁ weighted: 2D FLASH GE-image, TE 2.701 ms, TR 100 ms, 1 average, flip angle 30°, field of view 110×110 mm, 3 slices, slice thickness 1.0 mm, matrix size 256×256, bandwidth 40760.9 Hz, resolution 0.430×0.430×1 mm.
B₀-field maps: T23D TurboRARE, TR 20 ms, 2 TEs: 2.24 and 6.04 ms, flip angle 30°, field of view 102.5×102.5×102.5 mm, 1 slice, slice thickness 102.5 mm, matrix size 64×64×64, resolution 1.60×1.60×1.60 mm.
T₁ maps: 2D RARE VTR with 8 TRs: 3500, 2000, 1500, 1000, 800, 600, 400, 211.6 ms. Other parameters: TE 7 ms, field of view 48×48 mm, 8 slices, slice thickness 1 mm, matrix size 192×192, resolution 0.250×0.250×1 mm, bandwidth 75000 Hz, receiver gain 64.
T₂ weighted: 3D TurboRARE sequence, 16 TEs, TR 800 ms, flip angle 90°, field of view 51.2×51.2 mm, 2 slices, slice thickness 12.8 mm, matrix size 128×128×32, resolution 0.4×0.4×0.4 mm.
T₂ maps: 2D MSME sequence, 25 TEs ranging from 9 to 225 ms, TR 2000 ms, field of view 51.2×51.2 mm, 8 slices, slice thickness 1 mm, matrix size 256×256, resolution 0.20×0.20×1 mm.

To CALIBRATE the sample temperature via NMR spectrometric determination of the proton chemical shift difference between water and methanol (or ethylene glycol), the following sequence was adopted:
PRESS 1 H: TR 2500 ms, TE 16.6 ms, 128 averages, flip angle RF pulse 1: 90°, flip angle RF pulse 2: 180°, bandwidth 3301.6 Hz.

Image ANALYSIS: T₁and T₂ maps of phantoms were generated via the MRI Analysis Calculator tool with ImageJ and analysed by manually drawing a region of interest (ROIs) on phantom tubes of interest to quantify respective T₁and T₂ times. Additionally, images of the T₁and T₂ maps in grey and colour gradient were generated with ImageJ. Synthesis

THE synthesis of [Gd([¹⁹F]q-pH-1)] is shown in in reaction scheme 1 hereinafter. Reagents and conditions: (i) K₂CO₃, MeCN, RT; (ii) TFA (trifluoroacetic acid), RT; (iii) GdCl₃, aq. MOPS (3-(N-morpholino)propanesulfonic acid)/NaOH (pH 7.0), RT. Intermediate [3] was not isolated.

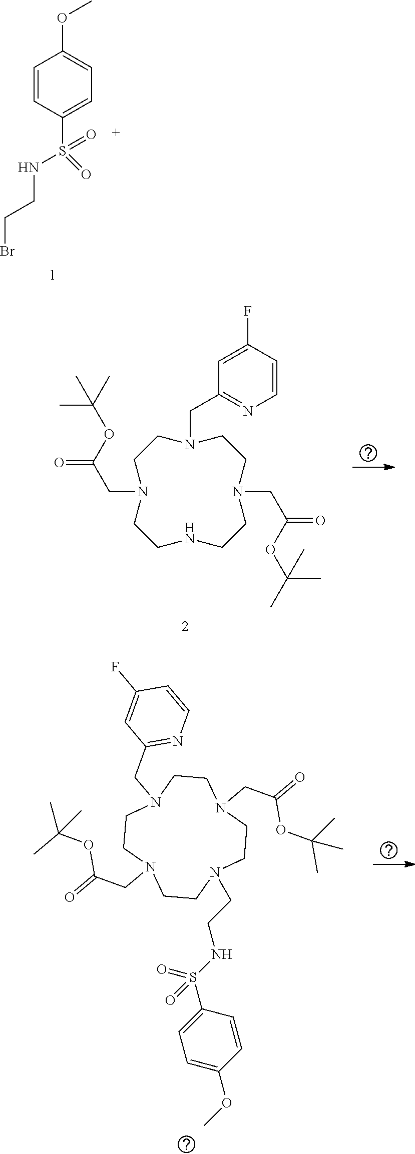

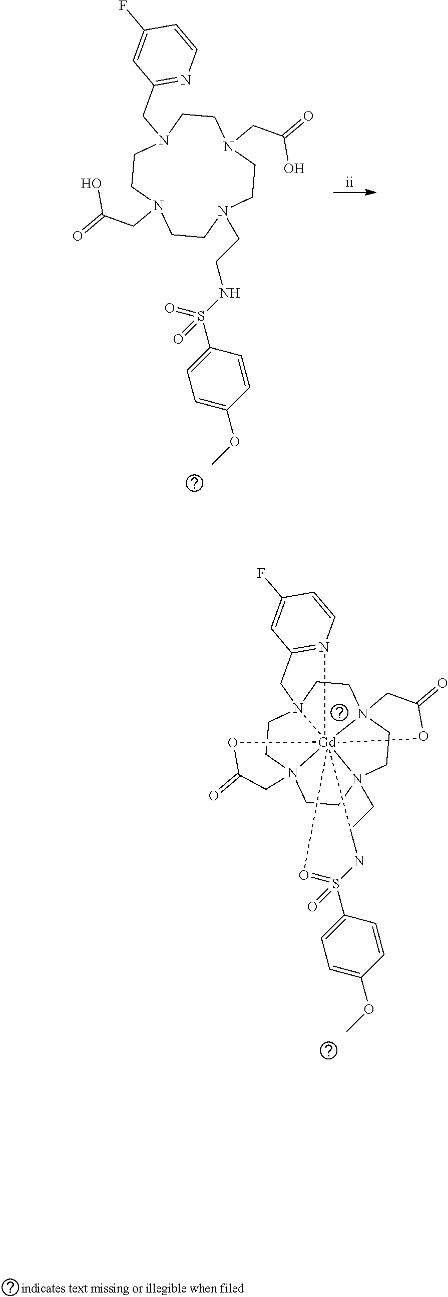

REACTION scheme 1. Synthesis of [Gd([¹⁹F]q-pH-1)].

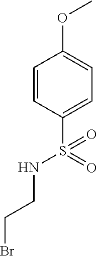

### SYNTHESIS OF N-(2-BROMOETHYL)-4-METHOXYBENZENESULFONAMIDE (1)

In a 50 mL round bottom glass flask, 4-Methoxybenzenesulfonyl chloride (1.00 g, 4.83 mmol, 1.0 equiv.) and 2-bromoethylamine hydrobromide (1.14 g, 5.55 mmol, 1.15 equiv.) were suspended in DCM (16.5 mL) at 0° C. Triethylamine (1.6 mL, 11.6 mmol, 2.4 equiv.) was added dropwise over the course of 10 minutes, and the resulting suspension was stirred for 1 h at RT. The completion of the reaction was confirmed with a TLC (50% EtOAc, 50% Methanol). The reaction mixture was diluted with DCM (20 mL) and washed with 1 M aqueous solution of HCl (2×20 mL) and with brine (2×20 mL). The organic phase was dried over Na₂SO₄ and, and liquids were evaporated on a rotary evaporator to yield the product as a white solid. Yield: 1.22 g (86%; 1 step; based on 4-Methoxybenzenesulfonyl chloride) NMR (CDCl₃): ¹H (500 MHz, T=300 K) δ_{H} 3.43-3.32 (CH₂—CH₂, m, 4H); 3.88 (CH₃, s, 3H); 5.20 (NH, br s, 1H); 6.99 (arom., d, J=9 Hz, 2H); 7.80 (arom., d, J 9 Hz, 2H).

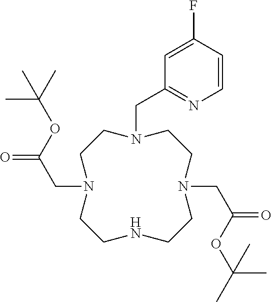 [SMILES: CC(C)(C)OC(=O)CN1CCNCCN(CC(=O)OC(C)(C)C)CCN(Cc2cc(F)ccn2)CC1]

### SYNTHESIS OF DI-TERT-BUTYL 2,2′-(4-((4-FLUOROPYRIDIN-2-YL)METHYL)-1,4,7,10-TETRAAZACYCLODODECANE-1,7-DIYL)DIACETATE (2)

A SOLUTION of (4-FLUOROPYRIDIN-2-YL) METHYL METHANESULFONATE (0.68 g, 3.32 mmol, 1.0 equiv.) in MeCN (160 mL) was added slowly dropwise to a solution of t-BuDO2A (2 g, 4.99 mmol, 1.5 equiv.) and K₂CO₃ (0.46 g, 3.32 mmol, 1.0 equiv.) in MeCN (340 mL). The resulting suspension was stirred for 48 hours at RT. The solids were filtered off and the filtrate was concentrated under reduced pressure. The residue was purified by flash column chromatography (C18, H₂O/MeCN gradient with 0.1% FA additive) to gave the product as a pale-yellow oil. Yield: 728 mg (42.9%, 1 step, based on (4-FLUOROPYRIDIN-2-YL) METHYL METHANESULFONATE). ¹H NMR (600 MHz, CD₃CN) 8.63 (dd, J=8.2, 5.9 Hz, 1H), 7.18 (dd, J=9.8, 1.6 Hz, 1H), 7.07 (ddd, J=8.4, 5.7, 2.5 Hz, 1H), 3.74 (s, 2H), 3.17 (s, 4H), 3.04 (t, 4H), 2.95 (t, 4H), 2.80 (t, J=4.9 Hz, 4H), 2.55 (t, 4H), 1.42 (s, 18H). LCMS: 510.30 [M+H]⁺ (theor. [C₂₆H₄₅O₄N₅F₁]⁺=510.35)

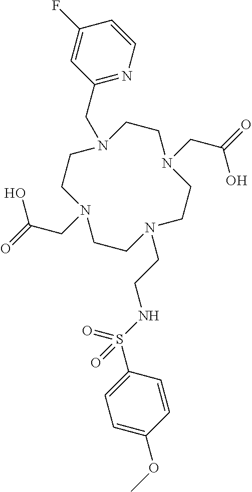 [SMILES: COc1ccc(S(=O)(=O)NCCN2CCN(CC(=O)O)CCN(Cc3cc(F)ccn3)CCN(CC(=O)O)CC2)cc1]

### SYNTHESIS OF LIGAND [¹⁹F]q-pH-1

A SOLUTION of 1 (64 mg, 0.22 mmol, 1.0 equiv.) in MeCN (10.5 mL) was prepared and cooled to 0° C. and slowly dropwise added via syringe over the course of 45 minutes to a suspension of 2 (137 mg, 0.22 mmol, 1.0 equiv.) and anhydrous K₂CO₃ (91 mg, 0.66 mmol, 3.0 equiv.) in MeCN (2.75 mL) cooled on ice bath to 0° C. The resulting suspension was stirred for 24 hours at RT. The solids were filtered off and the filtrate was concentrated under reduced pressure. The residue was suspended in 1.6 mL of 60% aqueous MeCN with a few drops of formic acid and purified using preparative HPLC (C18 column, H₂O/MeCN gradient containing 0.1% formic acid as an additive). Fractions containing pure product in the form of tert-butyl ester were pooled and lyophilized. The residue was dissolved in TFA (4 mL) and stirred for 24 h at RT. Volatiles were removed on a rotary evaporator and the residue was purified using preparative HPLC (C18, H₂O/MeCN gradient with 0.1% TFA additive). Fractions with the product were pooled and lyophilized to make a product in the form of TFA salt, a white solid. Yield: 81 mg (42%, 2 steps, based on 2-2.5FA assuming [¹⁹F]q-pH-1-2.5TFA, M_{R}=895.8). NMR (D₂O): ¹H (700 MHz, T=300 K) δ_{H} 1.69-3.76 (mc, CH₂—COOH, CH₂—CH₂—NHSO₂—Ar, m, 26H); 3.91 (CH₃, s, 3H); 7.06-7.10 (arom, m, 1H); 7.13-7.19 (arom, m, 3H); 7.80-7.85 (arom, m, 2H); 8.47 (arom, dd, 1 H, ⁴J_{HF}=8 Hz, ³J_{HH}=6 Hz). ¹³C{¹H} (176 MHz, T=300 K) 6c 38.9, 49.9, 52.4, 55.7, 58.7, 59.0, 62.5, 110.4, 110.5, 111.8, 111.9, 113.8, 114.7, 115.5, 117.1, 118.8, 129.2, 130.3, 152.3, 162.0, 162.7, 162.9, 163.1, 163.3, 168.3, 169.8, 179.5. ¹⁹F{¹H} (659 MHz, T=300.0 K) δ_{F} −103.2(s). LCMS: 611.25 [M+H]⁺ (theor. [C₂₇H₄₀O₇N₆F₁S₁]⁺=611.27).

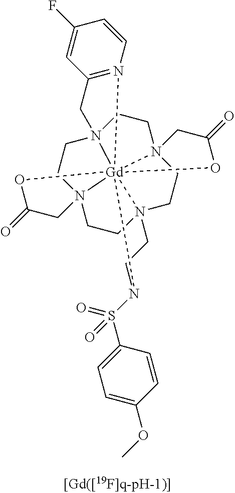

### SYNTHESIS OF [Gd([¹⁹F]q-pH-1)]

In a glass vial (25 mL), [¹⁹F]q-pH-1-2.5TFA (83 mg, 0.093 mmol, 1.0 equiv.) was dissolved in aq. MOPS/NaOH buffer (1 M, pH 7.0, 4 mL, 4.07 mmol, 44 equiv.) followed by adding aq. GdCl₃ (500 mM, 222 μL, 0.11 mmol, 1.2 equiv.) and the resulting solution was stirred at RT for 1 hour. The mixture was then purified by preparative HPLC (C18, H₂O/MeCN gradient with 0.1% AcOH additive). Fractions with the product were combined and lyophilized to give the product as a white solid. Yield: 76 mg (99%, 1 step, based on [¹⁹F]q-pH-1·2.5TFA assuming [Gd([¹⁹F]q-pH-1-1.0AcOH, M_{R}=826.0)). LCMS: 766.20 [M+H]⁺ (theor. [C₂₇H₃₇O₇N₆F₁S₁Gd]⁺=766.17).

The synthesis of [Gd(NO₂-q-pH-1)] is shown in reaction scheme 2 hereinafter. Reagents and conditions: (i) K₂CO₃, MeCN, RT; (ii) TFA (trifluoroacetic acid), RT; (iii) GdCl₃, aq. MOPS (3-(N-morpholino)propanesulfonic acid)/NaOH (pH 7.0), RT. The intermediate [5] was not isolated.

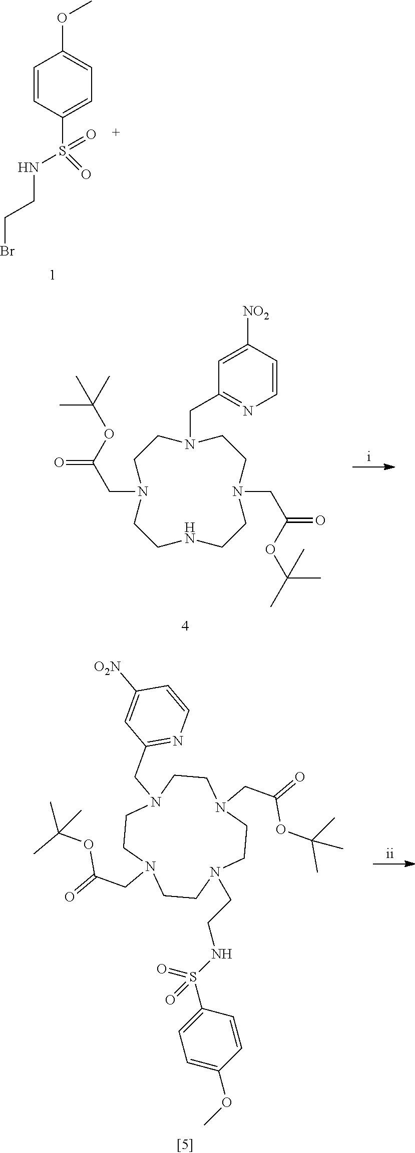

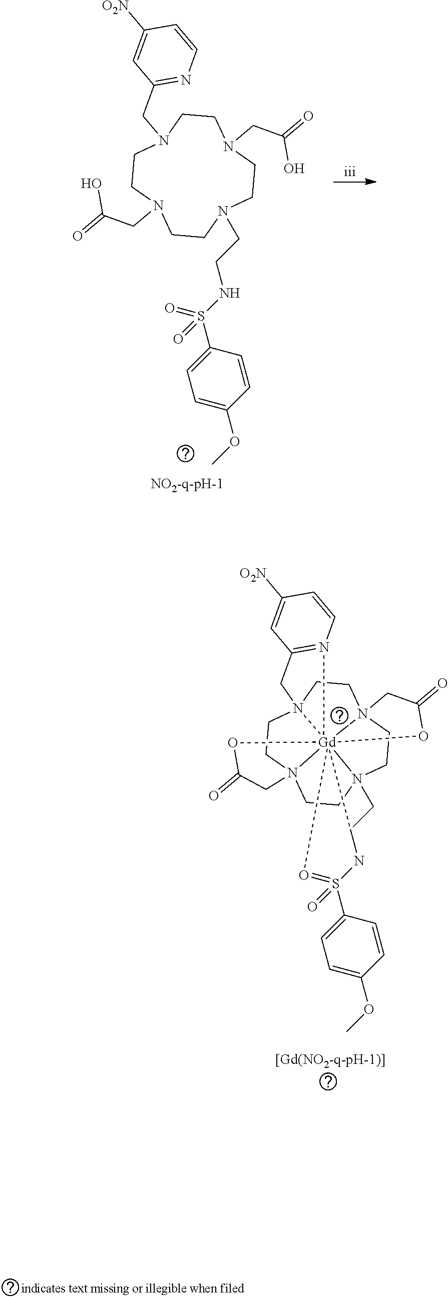

### Reaction Scheme 2. Synthesis of [Gd(NO₂-q-pH-1)]

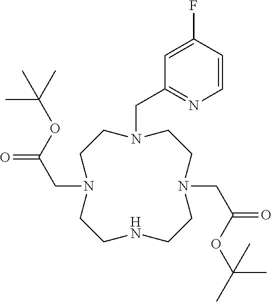 [SMILES: CC(C)(C)OC(=O)CN1CCNCCN(CC(=O)OC(C)(C)C)CCN(Cc2cc(F)ccn2)CC1]

### SYNTHESIS OF DI-TERT-BUTYL 2,2′-(4-((4-NITROPYRIDIN-2-YL)METHYL)-1,4,7,10-TETRAAZACYCLODODECANE-1,7-DIYL)DIACETATE (4)

(4-NITROPYRIDIN-2-YL)METHYL METHANESULFONATE (0.36 g, 1.53 mmol, 1.0 equiv.) in MeCN (80 mL) was added slowly dropwise to a solution of t-BuDO2A (0.92 g, 2.29 mmol, 1.5 equiv.) and K₂CO₃ (0.21 g, 1.53 mmol, 1.0 equiv.) in MeCN (150 mL). The resulting suspension was stirred for 48 hours at RT. The solids were filtered off and the filtrate was concentrated under reduced pressure. The residue was purified by flash column chromatography (C18, H₂O/MeCN gradient with 0.1% FA additive) to gave the product as a pale-yellow oil. Yield: 369 mg (45%, 1 step, based on (4-NITROPYRIDIN-2-YL)METHYL M ETHAN ESU LFONATE). ¹H NMR (600 MHz, CDCl₃) δ 1.37 (s, 18H), 2.23-3.48 (m, 22H), 3.84 (s, 2H), 7.84 (dd, J=5.4, 2.2 Hz, 1H), 8.67-8.93 (m, 2H). LCMS: 537.35 [M+H]⁺ (theor. [C₂₆H₄₅O₆N₆]⁺=537.34).

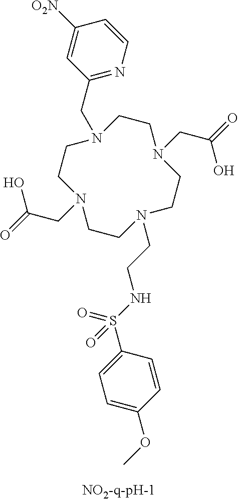 [SMILES: COc1ccc(S(=O)(=O)NCCN2CCN(CC(=O)O)CCN(Cc3cc([N+](=O)[O-])ccn3)CCN(CC(=O)O)CC2)cc1]

### SYNTHESIS OF LIGAND NO₂-q-pH-1

A solution of 1 (329 mg, 1.11 mmol, 1.2 equiv.) in MeCN (10.8 mL) was prepared and cooled to 0° C. and slowly dropwise added via syringe over the course of 45 minutes to a suspension of 4 (500 mg, 0.93 mmol, 1.0 equiv.) and anhydrous K₂CO₃ (515 mg, 3.73 mmol, 4.0 equiv.) in MeCN (2.8 mL) cooled on ice bath to 0° C. The resulting suspension was stirred for 16 hours at RT. The solids were filtered off and the filtrate was concentrated under reduced pressure. The residue was suspended in 1.6 mL of 60% aqueous MeCN with a few drops of formic acid and purified using preparative HPLC (C18 column, H₂O/MeCN gradient containing 0.1% formic acid as an additive). Fractions containing pure product in the form of tert-butyl ester were pooled and lyophilized. The residue was dissolved in TFA (4 mL) and stirred for 16 h at RT. Volatiles were removed on a rotary evaporator and the residue was purified using preparative HPLC (C18, H₂O/MeCN gradient with 0.1% TFA additive). Fractions with the product were pooled and lyophilized to make a product in the form of TFA salt, a white solid. Yield: 185 mg (22%, 2 steps, based on 4-2.5FA assuming NO₂-q-pH-1-2.5TFA, M_{R}=922). NMR (D₂O): ¹H (700 MHz, T=300 K) δ_{H} 2.54-3.84 (mc, CH₂—COOH, CH₂—CH₂—NHSO₂—Ar, m, 26H); 3.91 (CH₃, s, 3H); 7.15-7.21 (arom, m, 2H); 7.82-7.88 (arom, m, 2H); 8.19 (arom, dd, 1 H, ³J_{HH}=5 Hz, ⁴J_{HH}=5 Hz); 8.28 (arom, d, 1H, ⁴J_{HH}=2 Hz); 8.84 (arom, d, 1H, ³J_{HH}=6 Hz). ¹³C{¹H} (176 MHz, T=300 K) δ_{C} 37.5, 48.7, 48.9, 50.2, 51.4, 52.7, 53.9, 55.7, 57.3, 113.8, 114.9, 115.5, 116.8, 117.1, 117.2, 118.8, 128.7, 129.3, 151.8, 154.7, 162.6, 162.8, 163.0, 163.3, 173.7. LCMS: 638.25 [M+H]⁺ (theor. [C₂₇H₄₀O₉N₇S₁]⁺=638.26).

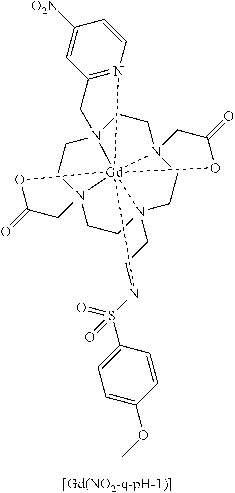

### SYNTHESIS AND LC-MS CHROMATOGRAM OF [Gd(NO₂-q-pH-1)]

IN a glass vial (4 mL), NO₂-q-pH-1-2.5TFA (22 mg, 0.024 mmol, 1.0 equiv.) was dissolved in aq. MOPS/NaOH buffer (1 M, pH 7.0, 4 mL, 0.73 mmol, 30 equiv.) followed by adding aq. GdCl₃ (500 mM, 63 μL, 0.032 mmol, 1.3 equiv.) and the resulting solution was stirred at RT for 1 hour. The mixture was then purified by preparative HPLC (C18, H₂O/MeCN gradient with 0.1% AcOH additive). Fractions with the product were combined and lyophilized to give the product as a white solid. Yield: 17 mg (81%, 1 step, based on NO₂-q-pH-1·2.5TFA assuming [Gd(NO₂-q-pH-1]-1.0AcOH, M_{R}=852.0)). LCMS: 793.20 [M+H]⁺ (theor. [C₂₇H₃₇O₉N₇S₁Gd]⁺=793.16).

### Radiolabeling Experiments

Radiolabelinq with ¹⁸F-Anion

### General Procedure

A solution containing ¹⁸F-produced on a cyclotron was absorbed on the QMA column (Sep-Pak, Waters) in a OTf-cycle and eluted with an eluting solution of 1 ml (5 mg TBAOTf in anhydrous methanol). A volume containing the desired activity was transferred to a 4 ml glass vial with a stirring bar preheated for 110° C. and a stream of argon was introduced. The aqueous solution was evaporated and 300 μl of dry MeCN was introduced and evaporated again. This step was performed twice. 100 μl of 12 mM solution of the [Gd(NO₂-q-pH-1] for radiolabeling in a dry DMSO was introduced, followed by the 2,5 μl of 1 M solution of a TBAF in a THF. The vial was transferred to an aluminium heat block preheated to 80° C. and the reaction mixture was stirred for 15 minutes. The reaction mixture was afterwards analysed on a HPLC equipped with the gamma and UV detector.

### Radiolabeling of [Gd(L¹)] by Nucleophilic Aromatic Substitution

[Gd(NO₂-q-pH-1] radiolabeling experiment is schematically depicted in reaction scheme 3 hereinafter.

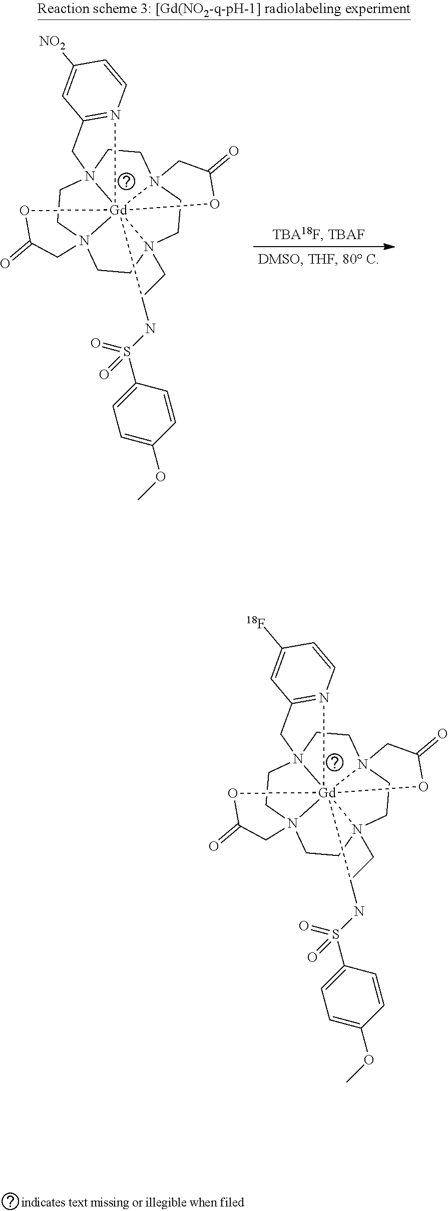

The results of the [Gd(NO₂-q-pH-1] radiolabeling experiment are shown in Table 1: Radiolabeling experiments were performed with [Gd(NO₂-q-pH-1] as a precursor. All the reactions were done in dry solvents. The amount of precursor used was 1 mg.

TABLE 1

| Experiment number | Radiochemical yield (%) |
|---|---|
| 1 | 44 |
| 2 | 27 |
| 3 | 41 |

A chromatogram from this radiolabeling experiment is depicted in FIG. 4. The radiochemical yield is defined as an amount of activity in the product expressed as the percentage of related starting activity.

In the gamma-chromatogram we observe unreacted [¹⁸F]fluoride with retention time of 1.599 min and product [¹⁸F]q-pH-1 with retention time of 9.300 min. The reaction proceeded with RCY of 44%.

### Imaging

Imaging—r₁ and r₂ relaxivity at 7T in different environments (HEPES, HSA, Human Serum)

For determination of r₁ and r₂ relaxivities, 5 samples of the contrast agent probe were prepared ranging between 0.1 and 0.5 mM concentrations in a HEPES buffer (10 mM, pH 7.0, 250 μL), 0.6 mM human albumin or human serum (Sigma-Aldrich). Additionally, 6 samples of the contrast agent were prepared with a constant concentration of 0.5 mM at different pH-levels (pH 5.0, 6.0, 6.5, 7.0 7.4, 8.0), adjusted via sodium hydroxide and hydrochloric acid, and dissolved in a HEPES buffer (10 mM, pH 7.0), human albumin (0.6 mM) or human serum (Sigma-Aldrich). The samples thus prepared were used for the determination of the T₁ and T₂ values on the 7T MR scanner at 21 and 37° C. using the above-described pulse sequences. Temperature was monitored using a spectroscopic PRESS sequence applied on a tube containing pure methanol or ethylene glycol, according to the manufacturer's protocol (Bruker).

The relaxivities r₁ and r₂ were determined as the slope from the plot of relaxation rates vs. concentration in mM.

Relative changes in relaxation rates (1/T₁) and (1/T₂) and ultimately in relaxivities in the pH range of interest (pH 6.5 to 7.4) were determined by plotting against different pH-levels and forming the ratio between relaxation rates at pH 6.5 and 7.4.

The r₁ and r₂ relaxivities of [Gd([¹⁹F]q-pH-1)] as well as relative drop in relaxation rates 1/T₁ and 1/T₂ within pH range of interest (pH 6.5 to 7.4) are shown in Table 2. Respectively measured at 7T in different buffering systems and at different temperatures.

TABLE 2

| | | | Change in | | Change in |
|---|---|---|---|---|---|
| | | r₁ [mM − 1*s − 1] | 1/T₁ [%] | r₂ [mM − 1*s − 1] | 1/T₂ [%] |
| HEPES | 21° C. | 1.47 | 14% | 3.55 | 6% |
| | 37° C. | 1.18 | 11% | 2.49 | 4% |
| HSA | 21° C. | 2.00 | 8-10% | 12.05 | no trend |
| | 37° C. | 1.68 | 8-10% | 10.16 | no trend |
| Human | 21° C. | 1.73 | 46% | 5.02 | 21% |
| Serum | 37° C. | 1.58 | 46% | 4.82 | 21% |

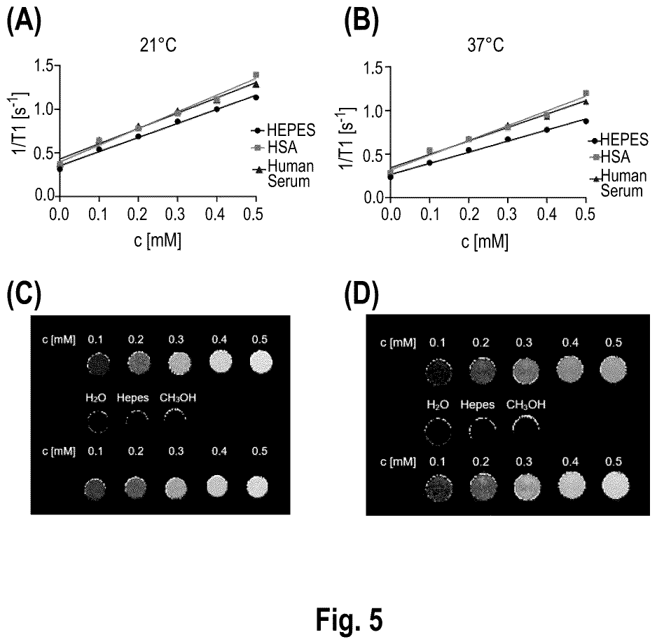

Longitudinal relaxation rates and MR imaging of [Gd([¹⁹F]q-pH-1)] in MR phantoms at 7T are shown with reference to FIG. 5. Figures A and B show relaxation rates (1/T₁) at 7T of the above-mentioned conditions of [Gd(q-pH0)] in 10 mM Hepes buffer, 0.6 mM human albumin or human serum, at 21° C. (a) or 37° C. (Figure B). FIGS. 5 C and D show T₁ maps of a MR phantom acquired at 21° C. for slice 4 (C) or 37° C. for slice 4 (FIG. 5 D). Signal intensities depicted in grey and colour gradients. A tube containing pure CH₃OH was used for temperature monitoring with MR spectroscopy.

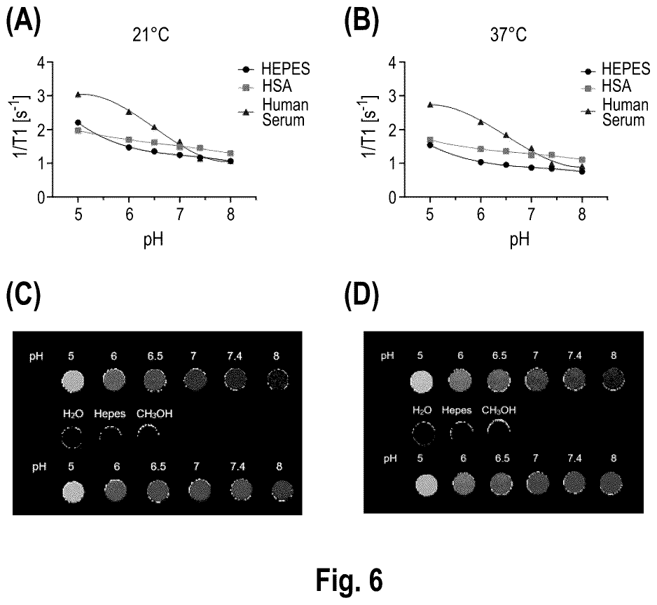

With reference to FIG. 6, the pH-dependent change in longitudinal relaxation rates and MR imaging of [Gd([¹⁹F]q-pH-1)] in MR phantoms at 7T is shown. FIGS. 6 A and B show telaxation rates (1/T₁) at 7T at above mentioned pH in 10 mM Hepes buffer, 0.6 mM human albumin or human serum, at 21° C. (a) or 37° C. (FIG. 6 B). FIGS. 6 C and D T₁ maps of a MR phantom acquired at 21° C. for slice 3 (FIG. 6 C) or 37° C. for slice 4 (FIG. 6 D). Signal intensities depicted in grey and colour gradients. A tube containing pure CH₃OH was used for temperature monitoring with MR spectroscopy.

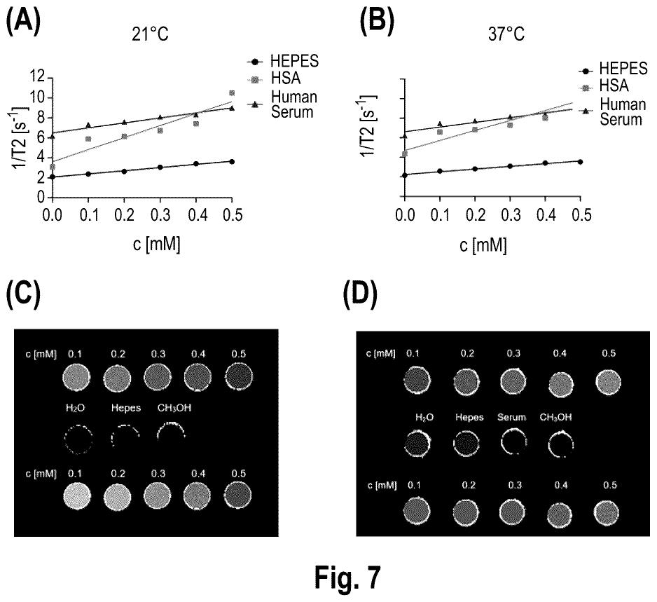

FIG. 7 shows the transversal relaxation rates and MR imaging of [Gd([¹⁹F]q-pH-1)] in MR phantoms at 7T. FIGS. 7A and B show relaxation rates (1/T₂) at 7T of above mentioned concentrations of [Gd(q-pH-1)] in 10 mM Hepes buffer, 0.6 mM human albumin or human serum, at 21° C. (a) or 37° C. (FIG. 7 B). FIGS. 7C and D show T₂ maps of a MR phantom acquired at 21° C. for slice 8 (C) or 37° C. for slice 5 (FIG. 7D). Signal intensities depicted in grey and colour gradients. A tube containing pure CH₃OH was used for temperature monitoring with MR spectroscopy.

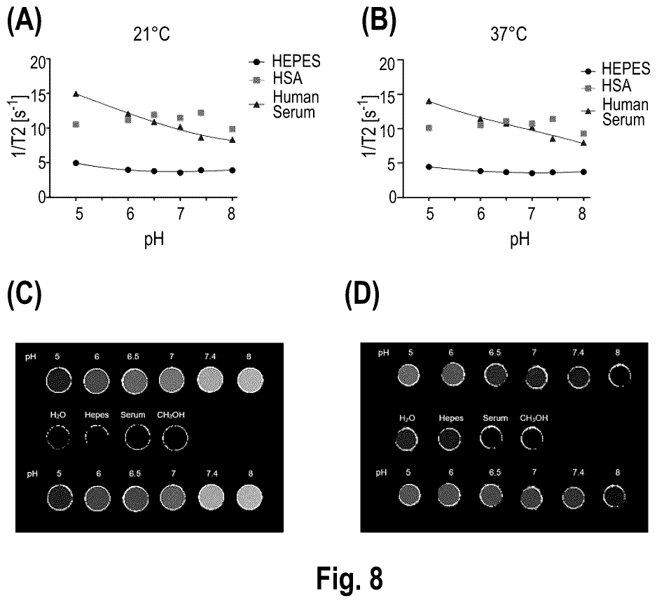

In FIG. 8 the pH-dependent change in transversal relaxation rates and MR imaging of [Gd([¹⁹F]q-pH-1)] in MR phantoms at 7T is shown. FIGS. 8 A and B show telaxation rates (1/T₂) at 7T at different pH in 10 mM Hepes buffer, 0.6 mM human albumin or human serum, at 21° C. (a) or 37° C. (FIG. 8 B). FIGS. 8C and D T₂ maps of a MR phantom acquired at 21° C. for slice 8 (C) or 37° C. for slice 5 (FIG. 8 D). Signal intensities depicted in grey and colour gradients. A tube containing pure CH₃OH was used for temperature monitoring with MR spectroscopy.

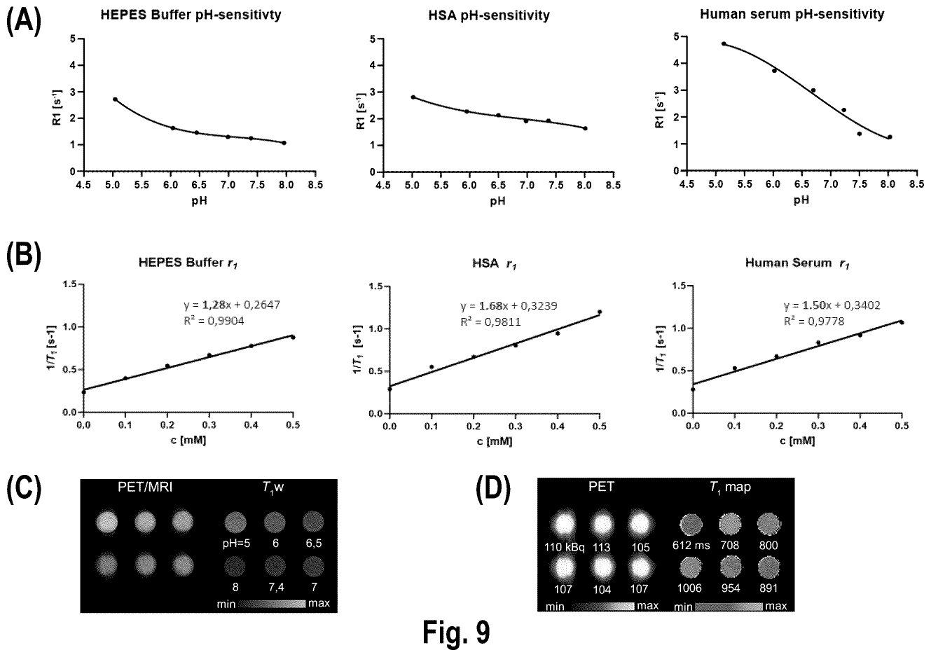

FIG. 9 shows in-vitro studies of q-pH-1. Respective relaxivities were determined in HEPES, HEPES/HSA, and human serum used 0.1-0.5 mM samples at pH 7.4m while pH-responsiveness between pH 5 and 8 was determined on a 7T MRI scanner. FIG. 9 A shows determination of [¹⁹F]q-pH-1 pH responsiveness at 37° C. in HEPES buffer, HEPES buffer with 0.6 mM HSA and in human serum on samples ranging from pH 5 to 8 and [¹⁹F]q-pH-1 concentration of 0.5 mM. In case in HEPES buffer pH is lowered from 7.4 to 6.5, R1 increase by 12% can be seen, in HEPES/HSA R1 increase by 8%, and in human serum increase by 55%. With reference to FIG. 9 B, it can be further seen that HEPES buffer has r₁=1.28 mM*s⁻¹, HSA r₁=1.68 mM*s⁻¹, and human serum r₁=1.50 mM*s⁻¹. In FIGS. 9 C and D six samples of constant concentrations of [¹⁹F]/[¹⁸F]-q-pH-1 in human serum at varying pH are shown. Simultaneous PET/MRI imaging on a 7T MRI scanner with PET insert is performed. The bimodal nature of the agent provides consistent PET signals, while the MRI clearly shows the response to different pH as can be derived from the T₁w and T₁map, respectively.

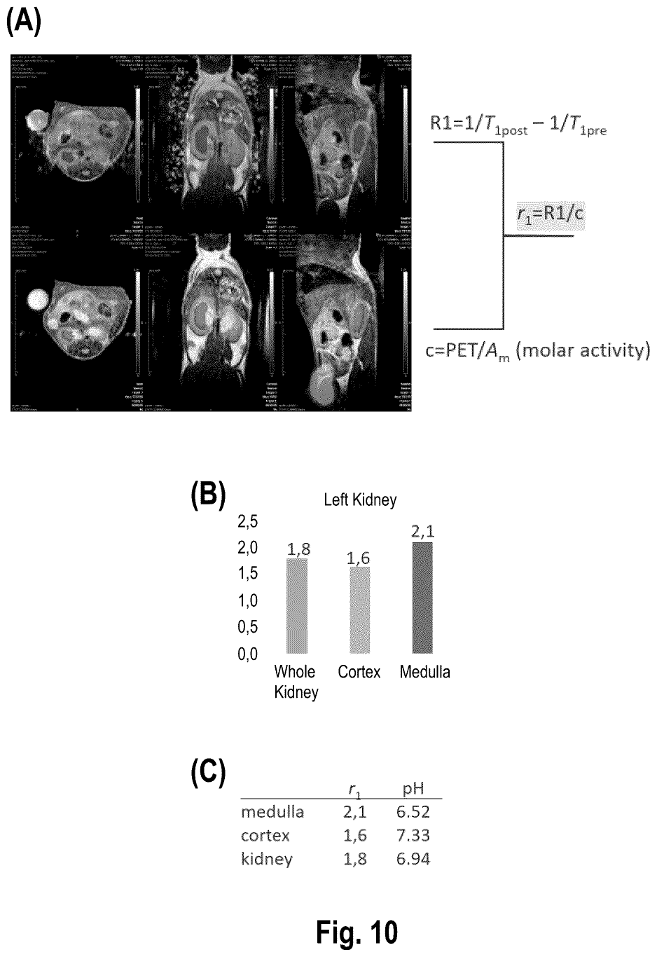

FIG. 10 shows q-pH-1 evaluation of different kidney regions in C57BL6 laboratory mice by simultaneous PET/MRI imaging in a 7T. Q-pH-1 at a concentration of 0.1 mmol/kg and 1 MBq has been used. FIG. 10A shows pH determination in two regions: cortex and medulla employing a modified Henderson-Hasselbalch equation, and pH=pKa+log₁₀ ((r₁−a)/b−r₁)) with pKa=6.55, a=r₁ at complete agent protonation, and b=r₁ at complete agent deprotonation. FIGS. 10 B and C indicate r₁ and pH values.

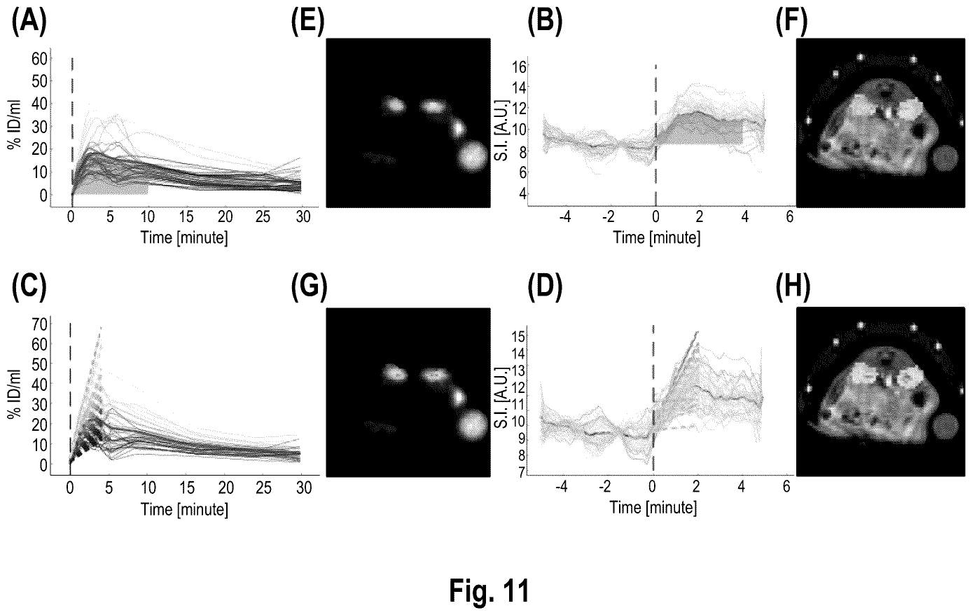

FIG. 11 shows kidneys perfusion evaluation using the present hybrid PET/MRI pH contrast agent. MRI S.I. (A.U.) and activity (% ID/mL) determined for each voxel in the ROIs were plotted over time to determine perfusion parameters such as area under the curve (AUC) and wash-in gradient. FIGS. 10 A-D show a voxel-wise dynamic representation of MR contrast and PET signal in kidneys. Dashed lines describe the linear regression used to calculate the wash-in parametric maps. FIGS. 10 E-F show AUC parametric maps obtained from dynamic curves and FIGS. 10 G-H wash-in parametric maps.

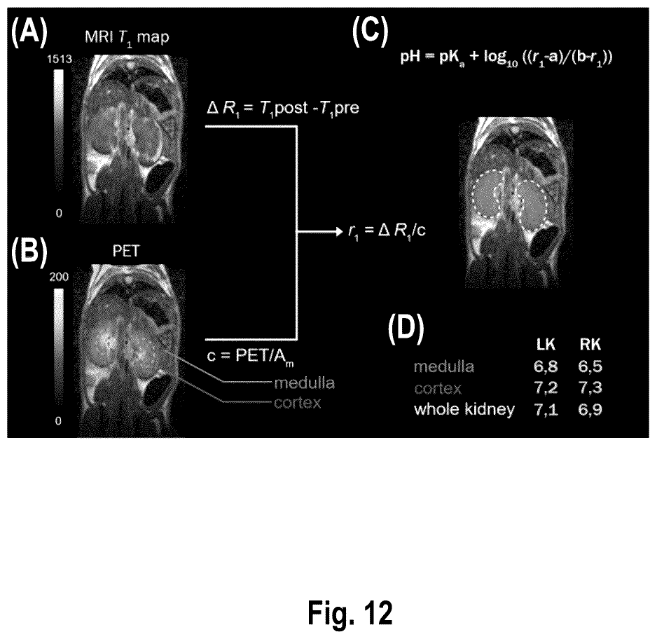

FIG. 12 shows in vivo pH evaluation. In FIG. 12 A MRI T1 maps are measured to calculate the relaxation rate (ΔR). PET is used to determine the in vivo agent concentration (FIG. 12 B). In FIG. 12 C the pH is calculated using the modified Henderson-Hasselbalch equation, where pKa represents the sulfonamide pendant arm and constants ‘a’ and ‘b’ represent the relaxivities of the fully protonated and deprotonated states. FIG. 12 D shows an evaluation of the pH of whole kidney, medulla and cortex.

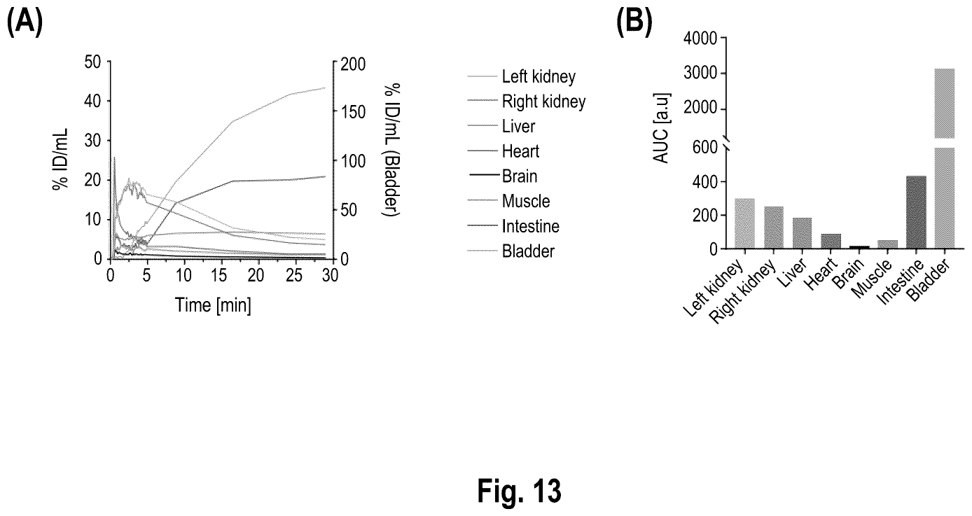

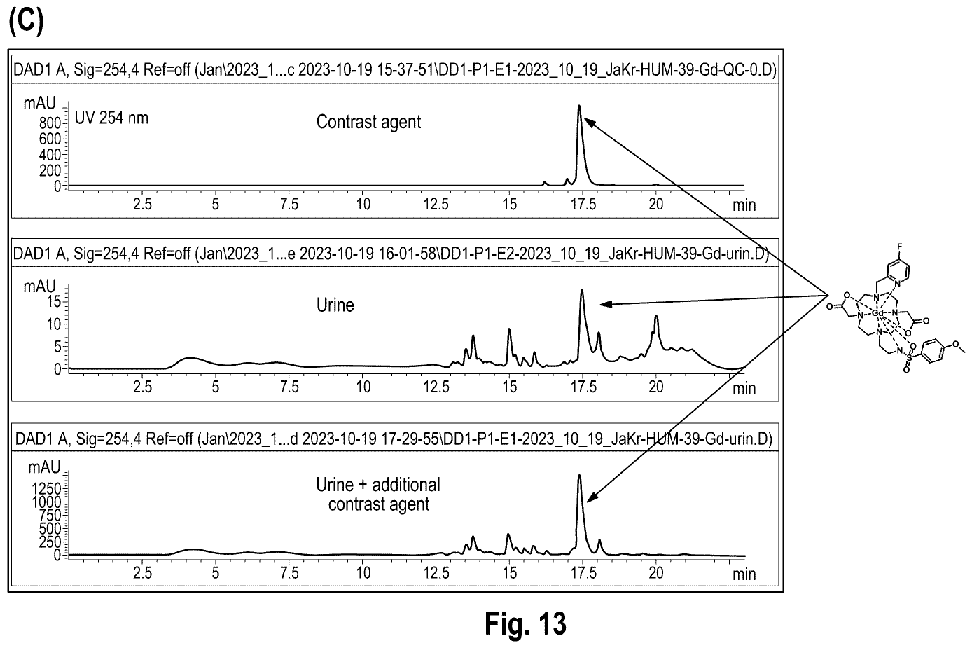

FIG. 13 shows the in-vivo PET pharmacokinetics of q-pH-1 in healthy mouse. PET-based pharmacokinetics were expressed as % ID/mL showing fast renal excretion (see FIG. 13 A). The biodistribution of the q-pH-1 determined from PET confirms the primarily renal excretion with minority excreted through the hepatobiliary system (see FIG. 13 B). Accordingly, q-pH-1 is predominantly eliminated via renal clearance, accompanied by partial hepatobiliary elimination. FIG. 13 C shows the HPLC analysis of stock solution (up), urine after in vivo experiment (middle) and urine with additional q-pH-1 (down). The HPLC analysis confirms that the metal complex stays intact during the in vivo experiment.

The above clearly demonstrates that the present compositions for imaging show a strong pH sensitivity, particularly in human serum. This makes q-pH-1 a promising tool for non-invasive pH quantification in vivo. The combination of PET and MRI provides a comprehensive view of tissue conditions. This multi-modal approach enhances precision di-agnostics, making it useful for personalized treatment strategies.

Q-pH-1 stability under physiological conditions and high contrast in imaging suggest a wide range of applications in oncology, neurology, and metabolic disease monitoring. In particular, q-pH-1 is capable of detecting and monitoring tumor progression in vivo due to the acidic microenvironment of many tumors. Since ischemic tissues undergo acidification, detecting and monitoring ischemia and stroke in vivo is possible as well. The same applies to inflammatory diseases that often correlate with pH shifts, which affect progression of the disease.

1. A composition for imaging, comprising
a compound of the general formula (I): 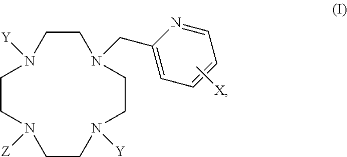
X is ¹⁸F, or ¹⁹F;
Y is independently selected from —(CH₂)ₙ—COOH or —(CH₂)ₙ—COO(CH₂)ₘY¹, wherein n is 1, 2 or 3, m is 0, 1 or 2, and Y¹ is C1 to C5 linear or branched alkyl; and
Z is —Z¹—NHSO₂—Z², Z¹ is linear or branched C1 to C5 alkyl or alkenyl, and Z² is substituted or unsubstituted alkyl, cyclyl or heterocyclyl;
or a salt thereof;
a lanthanide compound; and
optionally pharmaceutically acceptable excipients,
wherein a Lanthanide complex is formed between the compound of formula (I) or salt thereof and the Lanthanide compound.

2. The composition of claim 1, wherein X is in ortho or para position to N.

3. The composition of claim 1, wherein Y is —CH₂—COOH or —CH₂—COOCH₃.

4. The composition of claim 1, wherein Z¹ is —CH₄— or —CH₂—CH₄—.

5. The composition of claim 1, wherein Z² is phenyl having a single functional group in para position.

6. The composition of claim 1, wherein Z² is 4-methoxy-phenyl.

7. The composition of claim 1, wherein the Lanthanide compound is GdCl₃.

8. (canceled)

9. A method of using the composition of claim 1 for imaging, comprising the steps of:
(a) administering the composition to a subject in need thereof;
(b) detecting the Lanthanide complex; and
(c) forming an image therefrom.

10. The method of claim 9, wherein the composition is used for imaging an extracellular pH.

11. The method of claim 9, wherein the composition is used for imaging perfusion.

12. The method of claim 9, wherein the imaging is magnetic resonance imaging (MRI) and/or positron emission tomography (PET).

13. The method of claim 12, wherein magnetic resonance imaging (MRI) includes at least one of T₁ weighted imaging, T₂ weighted imaging, and T₂* weighted imaging.

14. The method of claim 9, wherein the subject is a vertebrate, a mammal or human.

15. The method of claim 9, wherein the composition is administered orally, systemically, via cerebral spinal fluid injection, vein injection, muscle injection, peritoneal injection, or subcutaneous injection.

16. The method of claim 9, wherein the imaging is in vivo.

17. (canceled)

18. A method of using the composition of claim 1 for imaging, comprising the steps of:
(a) contacting a target with the composition;
(b) detecting the Lanthanide complex; and
(c) measuring the amount and/or concentration of the Lanthanide complex in the target.

19. The method of claim 18, wherein the imaging is magnetic resonance imaging (MRI) and/or positron emission tomography (PET).

20. The method of claim 19, wherein magnetic resonance imaging (MRI) includes at least one of T₁ weighted imaging, T₂ weighted imaging, and T₂* weighted imaging.

21. The method of claim 18, wherein the target is blood or blood serum, bodily fluids, cerebrospinal fluid, tissue biopsy, cell, cell extract, organ and tissue.

22. (canceled)

23. The method of claim 18, the method further comprising (d) forming an image showing the amount and/or concentration of the Lanthanide complex in the target.

24.-35. (canceled)
